(19) United States
(12) Patent Application Publication
(10) Pub. No.: US 2005/0220732 A1
(43) Pub. Date: Oct. 6, 2005

(54) Novel polymers, compositions comprising them, processes therefor and use thereof

(76) Inventors: Timo Luukas, Massy (FR); Celine Farcet, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/090,270

(22) Filed: Mar. 28, 2005

Related U.S. Application Data
(60) Provisional application No. 60/560,271, filed on Apr. 8, 2004.

(30) Foreign Application Priority Data
Mar. 26, 2004 (FR) 0403183

(51) Int. Cl. A61K 7/42; A61K 7/04; A61K 7/021; A61K 7/06; A61K 7/11

(52) U.S. Cl. 424/61; 424/70.16; 424/70.7; 525/205; 424/63; 424/59

(57) ABSTRACT

Disclosed herein are novel block polymers comprising at least one monomer with an optical effect, and cosmetic compositions comprising the polymers, such as nail varnish compositions, foundation compositions, and anti-ageing compositions. Also disclosed herein are a cosmetic treatment process comprising applying to a keratin material the cosmetic composition as disclosed herein, and the use of these polymers as tensioning agents.

This application claims benefit of U.S. Provisional Application No. 60/560,271, filed Apr. 8, 2004, and French Patent Application No. 04 03183, filed Mar. 26, 2004, both of which are incorporated herein by reference.

The present disclosure relates to novel polymers of a particular structure and to cosmetic compositions comprising such polymers. The present disclosure also relates to a cosmetic treatment process using the polymers.

Various types of polymer are conventionally used in cosmetic compositions on account of the various properties that they can give to these compositions. They are used, for example, in makeup or care compositions for the skin, the lips or the integuments, such as nail varnishes or hair care compositions. However, in using two polymers that are incompatible, i.e., immiscible in the same solvent, within the same composition, the formulator is confronted, as a result of the incompatibility of the polymers, with problems of phase separation or even of decantation, and in general with the production of a non-uniform composition. These problems could only be solved hitherto by the presence in the composition of a compound for rendering the polymers mutually compatible.

Disclosed herein is a polymer which, when included in a composition, such as a cosmetic composition, enables this composition to avoid at least one of the drawbacks, limitations, defects and disadvantages of the compositions of the prior art.

The polymer as disclosed herein, referred to as a block polymer, comprises at least one first block and at least one second block that are incompatible with each other and that, for example, have different glass transition temperatures (Tg), wherein the at least one first block and the at least one second block are linked together via an intermediate segment comprising at least one constituent monomer of the first block and at least one constituent monomer of the second block.

Further disclosed herein is a composition, such as a cosmetic composition, comprising the polymer.

It has been found that the polymers as disclosed herein may have good optical properties that make it possible to use them in cosmetics, for obtaining adequate optical effects for the compositions comprising them and/or for the makeup obtained using these compositions.

Depending on the nature of the substituents, the polymers disclosed herein may show large variability in color, which may range from blue/violet to orange/red, passing through yellow. This makes it possible to have a range of compounds, belonging to the same chemical family and thus being formulated in a similar manner, and possessing a diversity of optical properties; this, for example, may facilitate the work of formulators by allowing them to keep a common architecture for all of their compositions, irrespective of the polymers with an optical property that are used.

Moreover, the polymers disclosed herein may have good fluorescence properties, and in some cases optical-brightening properties. The fluorescent compounds absorb in the ultraviolet and visible range, and re-emit energy by fluorescence with a wavelength ranging from 380 nm to 830 nm; when this wavelength ranges from 380 nm to 480 nm, i.e., in the blue region of the visible range, the compounds are then optical brighteners.

The polymers disclosed herein may be in solid or liquid form, and may give noteworthy optical effects to the compositions comprising them and also to the makeup applied; for example, the polymers disclosed herein can afford lightening effects, illuminating effects and/or color effects.

Moreover, these polymers can show good temperature, pH and light stability.

It has also been found that the polymers disclosed herein can show good solubility in fatty substances, it being possible for this solubility to vary and to be adjusted, according to the nature of the monomers. This good liposolubility may also facilitate their subsequent use, such as in cosmetic compositions generally comprising a fatty phase.

The term “at least one block” means one or more blocks.

The term “mutually incompatible blocks” means that the mixture formed from the polymer corresponding to the first block and from the polymer corresponding to the second block is immiscible in the polymerization solvent that is in weight majority for the block polymer, at room temperature (25° C.) and atmospheric pressure (10⁵ Pa), for a polymer mixture content of greater than or equal to 5% by weight relative to the total weight of the mixture (polymers and solvent), provided that:
i) the polymers are present in the mixture in a content such that the respective weight ratio ranges from 10/90 to 90/10, and that
ii) each of the polymers corresponding to the first block and the second block has an average (weight-average or number-average) molecular mass equal to that of the block polymer±15%.

In the case of a mixture of polymerization solvents, for example, when two or more solvents are present, the polymer mixture is immiscible in at least one of the solvents.

The intermediate segment is a block comprising at least one constituent monomer m1 of the first block and at least one constituent monomer m2 of the second block of the polymer; for example, m2 is different from m1. The intermediate segment or block may allow these first and second blocks to be “compatibilized.”

By incorporating these novel polymers into cosmetic compositions, the present inventors have discovered that such polymers may have very advantageous cosmetic properties.

In general, these polymers may increase the impact strength of nail varnishes and improve the staying power of a wide variety of makeup compositions, such as foundations or lipsticks, without causing the user any sensation of discomfort. The polymers may also have tensioning properties.

The block polymer disclosed herein is, for example, a linear block ethylenic polymer, for example, forming a deposit, such as a film-forming polymer.

The term “ethylenic polymer” means a polymer obtained by polymerization of monomers comprising at least one ethylenic unsaturation.

The term “block polymer” means a polymer comprising at least two different blocks such as at least 3 different blocks.

The polymer disclosed herein is a polymer of linear structure. In contrast, a polymer of non-linear structure is, for example, a polymer of branched, starburst or grafted structure.

The term “polymer forming a deposit” means a polymer capable, by itself or in the presence of an auxiliary agent, of forming a deposit that adheres to a support such as a keratin material.

The term “film-forming polymer” means a polymer that is capable, by itself or in the presence of an auxiliary film-forming agent of forming a continuous film that adheres to a support such as a keratin material.

The polymer disclosed herein comprises at least one first block and at least one second block that are incompatible with each other and that, for example, have different glass transition temperatures (Tg), wherein the at least one first block and the at least one second block are linked together via an intermediate segment comprising at least one constituent monomer of the first block and at least one constituent monomer of the second block.

As used herein, the terms “first” and “second” blocks do not in any way limit the order of the blocks in the structure of the polymer.

Each block of the polymer disclosed herein is derived from one type of monomer or from several different types of monomers. This means that each block may comprise at least one homopolymer or at least one copolymer; this at least one copolymer constituting the block may in turn be random or alternating.

For example, the intermediate segment comprising at least one constituent monomer of the first block and at least one constituent monomer of the second block of the polymer is a random polymer. In one embodiment, the intermediate block is essentially derived from constituent monomers of the first block and of the second block. The term “essentially” means at least 85%, such as at least 90%, further such as at least 95% and even further such as 100%.

In one embodiment, the intermediate block has a glass transition temperature Tg that is between the glass transition temperatures of the first and second blocks.

According to the present disclosure, the first and second blocks have, for example, different glass transition temperatures, with a difference between the glass transition temperatures of the first and second blocks generally of, for example, greater than 5° C., such as greater than 10° C. and further such as greater than 20° C.

The glass transition temperatures indicated for the first and second blocks may be theoretical Tg values determined from the theoretical Tg values of the constituent monomers of each of the blocks, which may be found in a reference manual such as the Polymer Handbook, 3rd Edition, 1989, John Wiley, according to the following relationship, known as Fox's law:

$$\frac{1}{Tg} = {\sum }_{i} ( \frac{\overline{ω} ⅈ}{Tgi} )$$

wherein \overline{ω}i is the mass fraction of the monomer i in the block under consideration and Tgi is the glass transition temperature of the homopolymer of the monomer i.

Unless otherwise indicated, the Tg values indicated for the first and second blocks in the present disclosure are theoretical Tg values.

In all cases, at least one of the blocks of the polymer disclosed herein comprises a monomer hereinbelow referred to as a monomer “with an optical effect”, which is of formula (I): 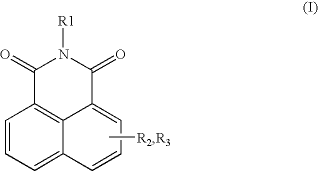
wherein:
R1 is chosen from linear, branched and cyclic, saturated and unsaturated carbon-based radicals comprising from 1 to 32 carbon atoms; optionally substituted with at least one substituent chosen from ═O, OH, NH₂ and halogen atoms; and/or optionally interrupted with at least one hetero atom chosen from O, N, P, Si and S;
R₂ and R₃, which may be identical or different and are present on the same ring or each on a different ring, are each chosen from a hydrogen atom, halogen atoms, and a group of formula —X-G-P₁ (II), with a proviso that at least one of the radicals R₂ and R₃ is a group of formula (II), wherein:
   X is chosen from the groups —O—, —S—, —SO—, —SO₂—, —NH— and —NR₄— with R₄ be chosen from linear, branched and cyclic, saturated and unsaturated carbon-based radicals comprising from 1 to 30 carbon atoms, optionally substituted with at least one substituent chosen from ═O, OH, NH₂ and halogen atoms; and/or optionally interrupted with at least one hetero atom chosen from O, N, P, Si and S;
   G is chosen from linear, branched and cyclic, saturated and unsaturated divalent carbon-based radicals comprising from 1 to 32 carbon atoms, optionally substituted with at least one substituent chosen from ═O, OH, NH₂ and halogen atoms; and/or optionally interrupted with at least one hetero atom chosen from O, N, P, Si and S;
   P₁ is a polymerizable group chosen from groups of the following formulae: 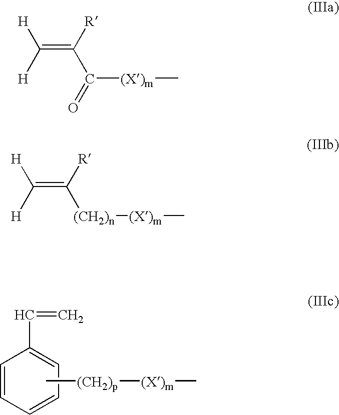
   wherein:
      R′ is chosen from H and linear and branched, saturated C₁₋₆ hydrocarbon-based radicals,
      X′ is chosen from O, NH and NR″ with R″ being a radical chosen from C₁₋₆ alkyl, C₆₋₁₀ aryl, (C₆₋₁₀)aryl(C₁₋₆)alkyl and (C₁₋₆)alkyl(C₆₋₁₀)aryl radicals, wherein at least one of the alkyl and aryl groups may be substituted with at least one substituent chosen from OH, halogen atoms, C₁₋₆ alkoxy and C₆₋₁₀ aryloxy groups; and
      m is equal to 0 or 1; n is equal to 0 or 1; and p is equal to 0, 1 or 2.

As used herein, the term “cyclic radical” means a monocyclic or polycyclic radical, which is itself in the form of one or more saturated and/or unsaturated, optionally substituted rings (for example, cyclohexyl, cyclodecyl, benzyl or fluorenyl), but also a radical comprising at least one of the rings (for example, p-tert-butylcyclohexyl or 4-hydroxybenzyl).

Further as used herein, the term “saturated and/or unsaturated radical” means totally saturated radicals, totally unsaturated radicals, including aromatic radicals, and also radicals comprising one or more double and/or triple bonds, the rest of the bonds being single bonds.

For example, R1 may be chosen from cyclic, linear and branched, saturated and unsaturated hydrocarbon-based radicals, optionally comprising a hydrocarbon-based ring that is itself saturated or unsaturated, comprising from 3 to 18 such as from 4 to 14 carbon atoms, and may comprise at least one hetero atom chosen, for example, from nitrogen, oxygen and silicon atoms.

Further, for example, R1 may be chosen from linear, branched and cyclic, saturated and unsaturated hydrocarbon-based radicals comprising from 6 to 13 carbon atoms.

In one embodiment, R1 is chosen from n-propyl, isopropyl, n-butyl, isobutyl, tert-butyl, n-pentyl, cyclopentyl, n-hexyl, cyclohexyl, n-heptyl, n-octyl, cyclooctyl, decyl, cyclodecyl, dodecyl, cyclododecyl, p-tert-butylcyclohexyl, benzyl and phenyl radicals, ethyl-2-N-pyrrolidine, (2-methyl)-1-ethylpyrrolidine, 3-propyltriethoxysilane, dialkylpyrimidines such as 2-(4,6-dimethyl)pyrimidine, benzothiazyl, and fluorenyl radicals.

| | |
|---|---|
| 2-methyl-1-ethylpyrrolidine | 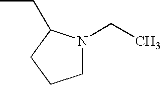 |
| ethyl-2-N-pyrrolidine | 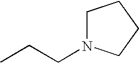 |
| 3-propyltriethoxysilane | 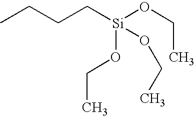 |
| benzothiazyl | 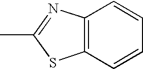 |
| fluorenyl | 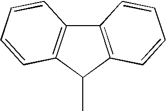 |
| 2-(4,6-dimethyl)pyrimidine | 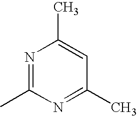 |

The radical R₂ is, for example, a hydrogen atom, and R₃ is therefore a group of formula (II).

In the group of formula (II), X is, for example, chosen from —O—, —S—, —NH— and —NR₄—.

The radical R₄ is, for example, chosen from linear, branched and cyclic, saturated and unsaturated, hydrocarbon-based radicals optionally comprising a hydrocarbon-based ring that is itself saturated or unsaturated, comprising from 2 to 18 such as from 3 to 12 carbon atoms, optionally substituted with at least one substituent chosen from ═O, OH, NH₂ and halogen atoms; and/or optionally interrupted with at least one hetero atom chosen from O, N, P, Si and S.

R₄ may, for example, be chosen from ethyl, n-propyl, isopropyl, n-butyl, isobutyl, tert-butyl, pentyl, hexyl, cyclohexyl, octyl, decyl, dodecyl, phenyl and benzyl radicals.

The divalent radical G is, for example, chosen from linear, branched and cyclic, saturated and unsaturated divalent hydrocarbon-based radicals, optionally comprising a hydrocarbon-based ring that is itself saturated or unsaturated, comprising in total from 2 to 18 such as from 3 to 8 carbon atoms, optionally substituted with at least one substituent chosen from ═O, OH, NH₂ and halogen atoms; and/or optionally interrupted with at least one hetero atom chosen from O, N, P, S and Si.

Further, for example, G is chosen from linear and branched, saturated divalent hydrocarbon-based radicals optionally comprising a saturated hydrocarbon-based ring, comprising in total from 2 to 16 such as from 3 to 10 carbon atoms.

In one embodiment, G is chosen from ethylene, n-propylene, isopropylene (1-methylethylene and 2-methylethylene), n-butylene, isobutylene, pentylene, such as n-pentylene, hexylene, such as n-hexylene, heptylene, cyclohexylene, octylene, decylene, cyclohexyldimethylene, such as of the formula —CH₂—C₆H₁₀—CH₂—, and dodecylene.

In the formula (IIIb), if n=0, then, for example, m=0.

The polymerizable group P₁ is chosen, for example, from groups of the following formulae: 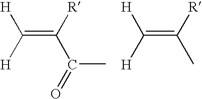
wherein R′ is H or methyl.

Among the monomer compounds that may be used herein, mention may be made, for example, of the compounds corresponding to one of the following formulae:

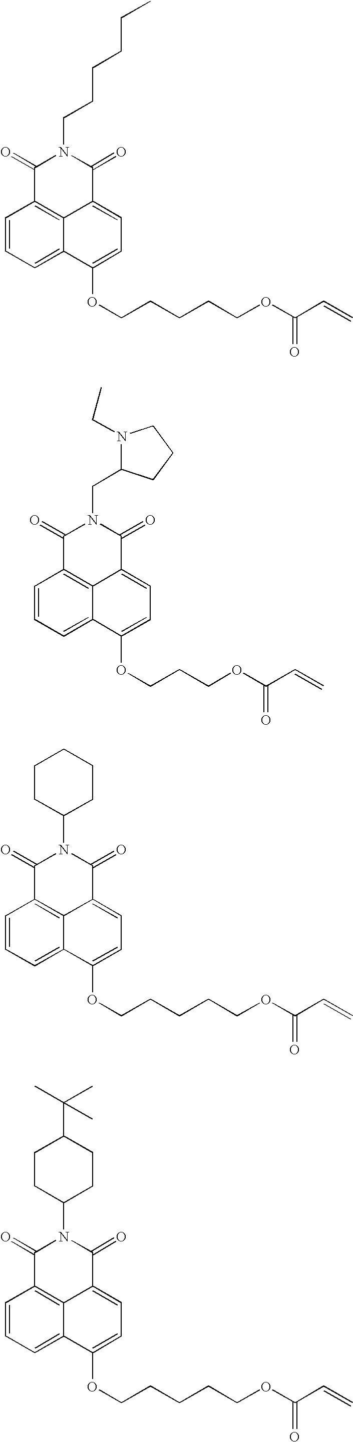

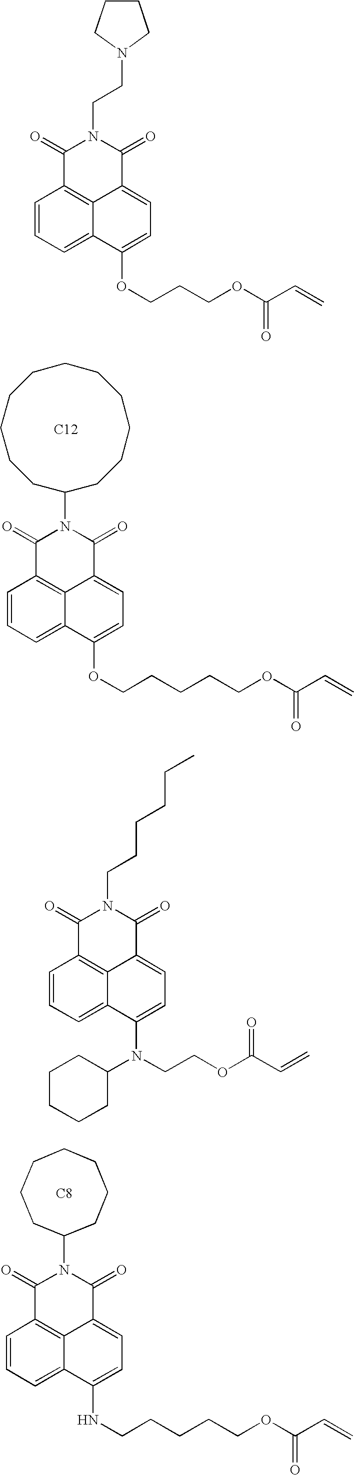

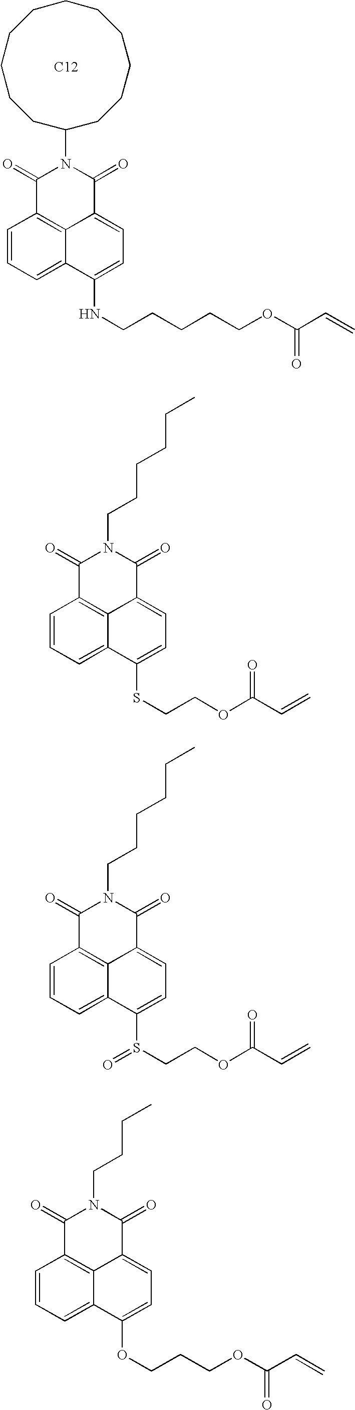

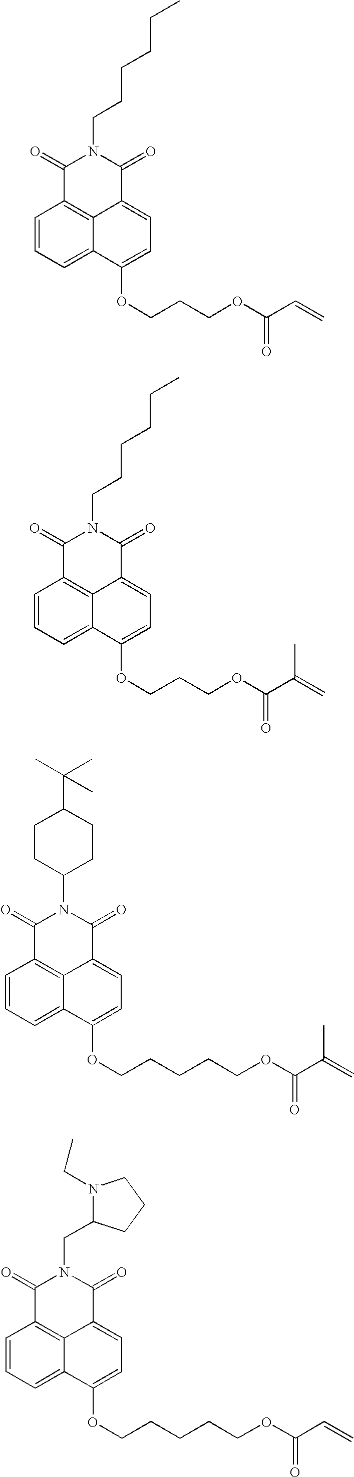

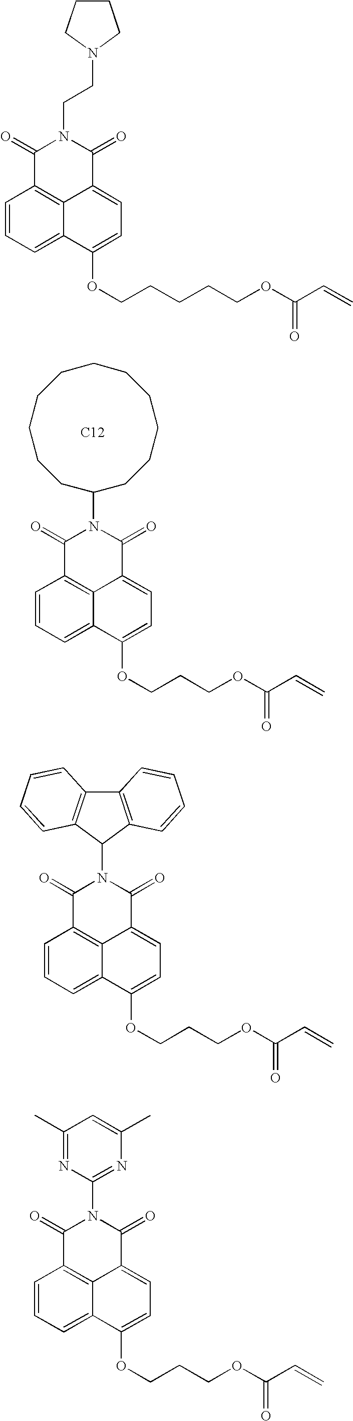

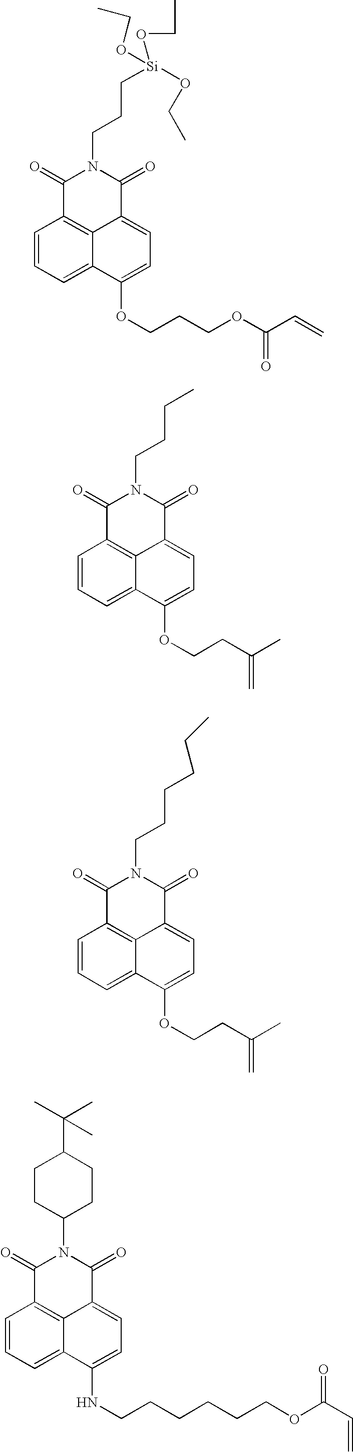

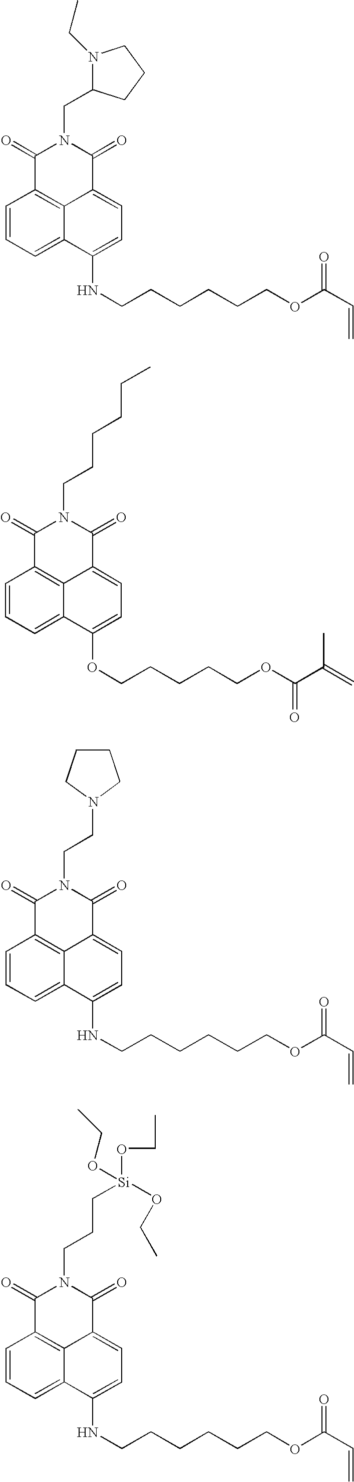

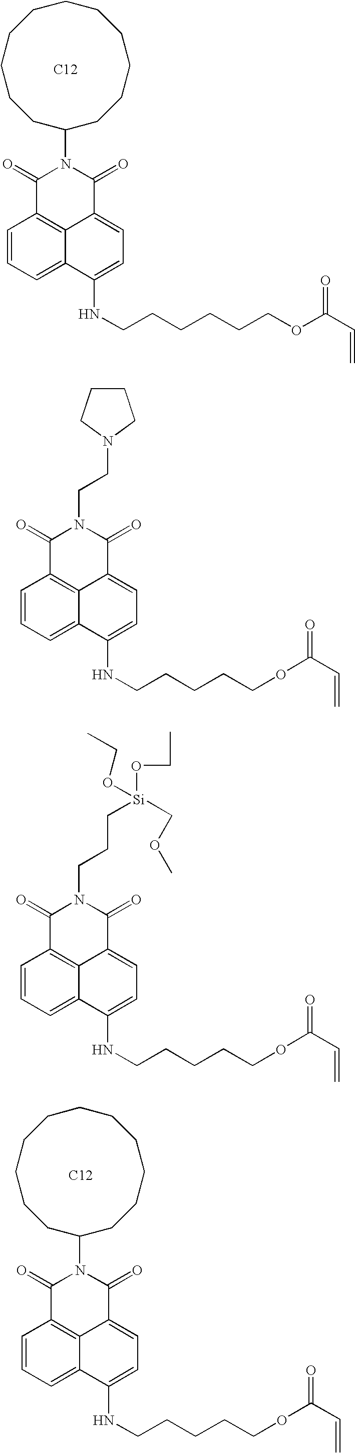

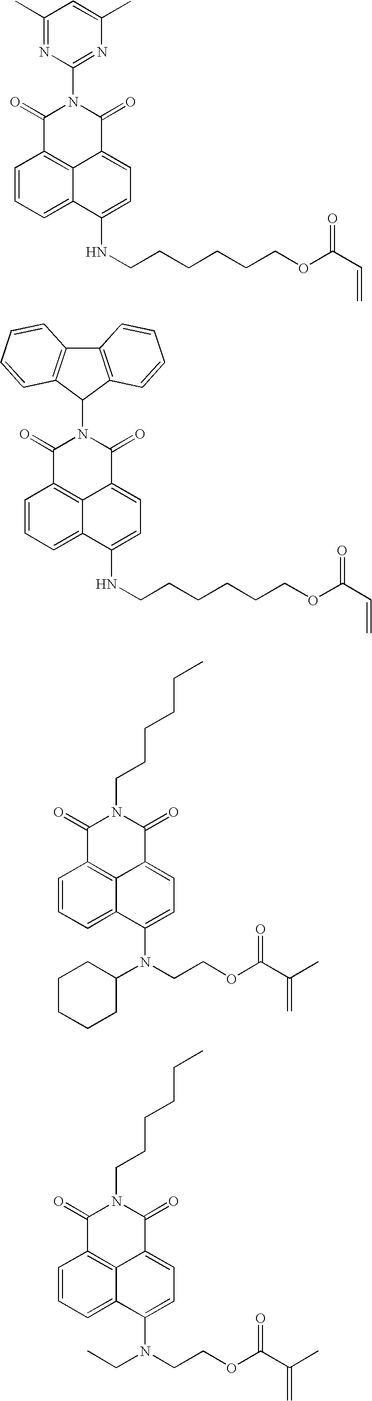

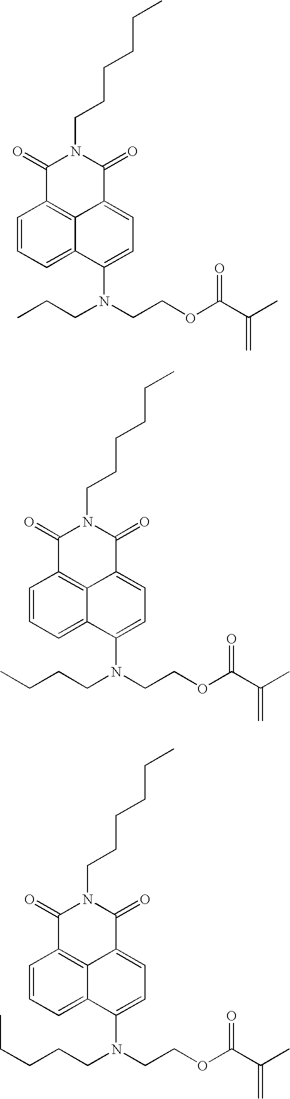

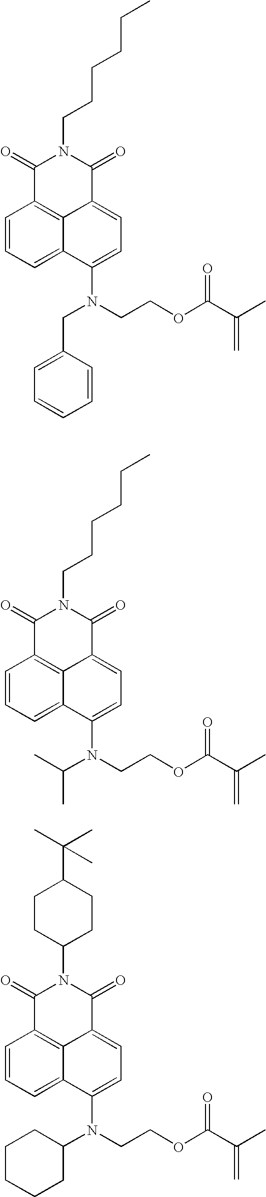

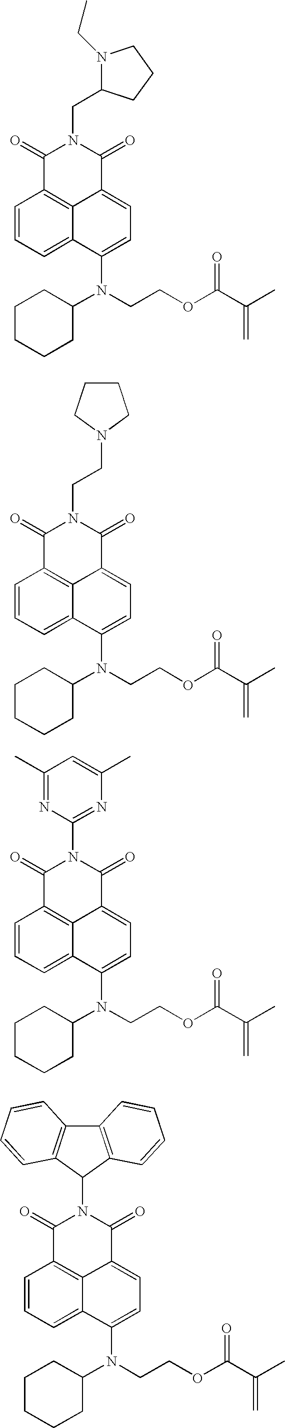

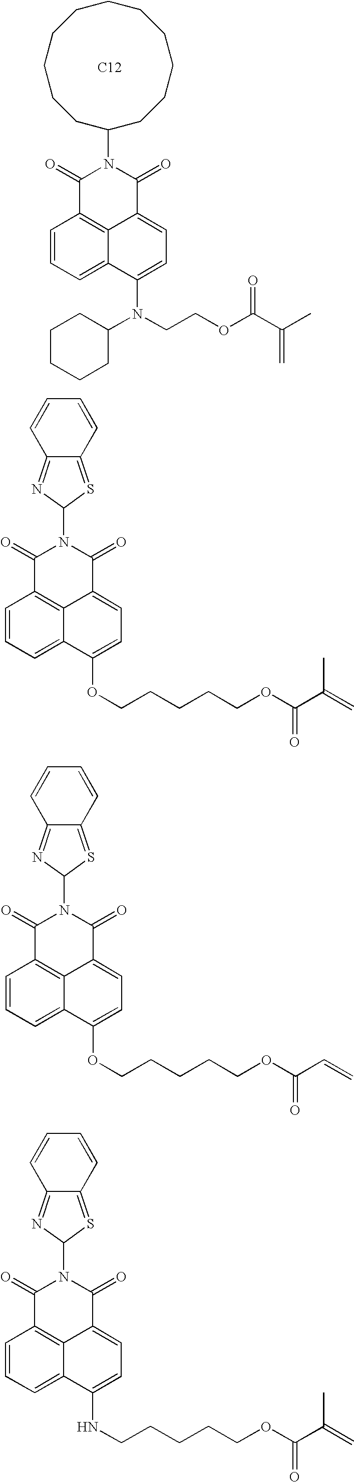

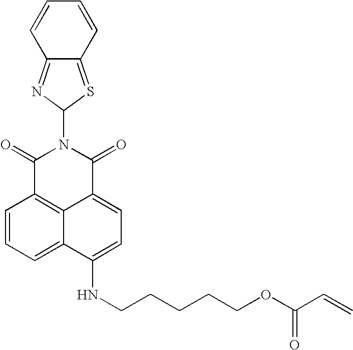

The monomer may be present, alone or as a mixture, in at least one of the first and the second blocks.

The block comprising at least one monomer with an optical effect of formula (I) may be, for example:
(i) a homopolymer comprising only one monomer with an optical effect of formula (I),
(ii) a copolymer comprising more than one monomer with an optical effect of formula (I),
(iii) a copolymer comprising one or more monomers with an optical effect of formula (I), and one or more additional monomers, which may be chosen, for example, from the monomers with an optical effect of formulae (A), (B) and (C) below and the “usual” additional monomers.

Among the “usual” additional monomers that may be present in the block comprising at least one monomer with an optical effect of formula (I), and/or that may be present in at least one other block not comprising a monomer with an optical effect of formula (I), mention may be made, for example, alone or as a mixture, of the following monomers:
(i) ethylenic hydrocarbons comprising from 2 to 10 carbons, such as ethylene, isoprene or butadiene;
(ii) the (meth)acrylates of formula: 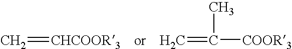
   wherein R′₃ is chosen from:
   linear and branched alkyl groups comprising from 1 to 18 carbon atoms, in which is optionally intercalated at least one hetero atom chosen from O, N, S and P; wherein the alkyl groups may optionally be substituted with at least one substituent chosen from a hydroxyl group, halogen atoms (Cl, Br, I and F), and groups Si(R₄R₅), wherein R₄ and R₅, which may be identical or different, are each chosen from C₁ to C₆ alkyl groups and a phenyl group;
   R′₃ may, for example, be chosen from methyl, ethyl, propyl, n-butyl, isobutyl, tert-butyl, hexyl, ethylhexyl, octyl, lauryl, isooctyl, isodecyl, dodecyl, cyclohexyl, t-butylcyclohexyl and stearyl groups; 2-ethylperfluorohexyl and C₁₋₄ hydroxyalkyl groups such as 2-hydroxyethyl, 2-hydroxybutyl and 2-hydroxypropyl; and (C₁₋₄)alkoxy(C₁₋₄)alkyl groups such as methoxyethyl, ethoxyethyl and methoxypropyl,
   C₃ to C₁₂ cycloalkyl groups such as an isobornyl group,
   C₃ to C₂₀ aryl groups such as a phenyl group,
   C₄ to C₃₀ aralkyl groups (C₁ to C₈ alkyl groups) such as 2-phenylethyl, t-butylbenzyl and benzyl,
   4- to 12-membered heterocyclic groups comprising at least one hetero atom chosen from O, N and S, the ring being aromatic or non-aromatic,
   heterocycloalkyl groups (C₁ to C₄ alkyl groups), such as furfurylmethyl and tetrahydrofurfurylmethyl,
   wherein at least one of the cycloalkyl, aryl, aralkyl, heterocyclic and heterocycloalkyl groups may optionally be substituted with at least one substituent chosen from a hydroxyl group, halogen atoms, and linear and branched C₁₋₄ alkyl groups in which is optionally intercalated at least one hetero atom chosen from O, N, S and P, wherein the alkyl groups may also optionally be substituted with at least one substituent chosen from a hydroxyl group, halogen atoms (Cl, Br, I and F), and groups Si(R₄R₅), wherein R4 and R₅, which may be identical or different, are each chosen from C₁ to C₆ alkyl groups and a phenyl group,
   R′₃ may also be a group —(C₂H₄O)ₘ—R″, with m=5 to 150 and R″=H or C₁ to C₃₀ alkyl, for example, —POE-methyl or —POE-behenyl;
(iii) the (meth)acrylamides of formula: 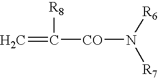
wherein R₈ is H or methyl; and R₆ and R₇, which may be identical or different, are each chosen from:
   a hydrogen atom; and
   linear and branched alkyl groups comprising from 1 to 18 carbon atoms, in which is optionally intercalated at least one hetero atom chosen from O, N, S and P; wherein the alkyl groups may also optionally be substituted with at least one substituent chosen from a hydroxyl group, halogen atoms (Cl, Br, I and F), and groups Si(R₄R₅), wherein R₄ and R₅, which may be identical or different, are each chosen from C₁ to C₆ alkyl groups and a phenyl group;
   at least one of R₆ and R₇ may, for example, be chosen from methyl, ethyl, propyl, n-butyl, isobutyl, tert-butyl, hexyl, ethylhexyl, octyl, lauryl, isooctyl, isodecyl, dodecyl, cyclohexyl, t-butylcyclohexyl and stearyl groups; 2-ethylperfluorohexyl and C₁₋₄ hydroxyalkyl groups such as 2-hydroxyethyl, 2-hydroxybutyl and 2-hydroxypropyl; and (C₁₋₄)alkoxy(C₁₋₄)alkyl groups such as methoxyethyl, ethoxyethyl and methoxypropyl,
   C₃ to C₁₂ cycloalkyl groups, such as an isobornyl group,
   C₃ to C₂₀ aryl groups such as a phenyl group,
   C₄ to C₃₀ aralkyl groups (C₁ to C₈ alkyl groups) such as 2-phenylethyl, t-butylbenzyl and benzyl,
   4- to 12-membered heterocyclic groups comprising at least one hetero atom chosen from O, N and S, the ring being aromatic or non-aromatic,
   heterocycloalkyl groups (C₁ to C₄ alkyl groups), such as furfurylmethyl and tetrahydrofurfurylmethyl,
   wherein at least one of the cycloalkyl, aryl, aralkyl, heterocyclic and heterocycloalkyl groups may optionally be substituted with at least one substituent chosen from a hydroxyl group, halogen atoms, and linear and branched C₁-C₄ alkyl groups in which is optionally intercalated at least one hetero atom chosen from O, N, S and P, wherein the alkyl groups may optionally be substituted with at least one substituent chosen from a hydroxyl group, halogen atoms (Cl, Br, I and F), and groups Si(R₄R₅), wherein R₄ and R₅, which may be identical or different, are each chosen from C₁ to C₆ alkyl groups and a phenyl group.

Examples of the (meth)acrylamide monomers include (meth)acrylamide, N-ethyl(meth)acrylamide, N-butylacrylamide, N-t-butylacrylamide, N-isopropylacrylamide, N,N-dimethyl(meth)acrylamide, N,N-dibutylacrylamide, N-octylacrylamide, N-dodecylacrylamide, undecylacrylamide and N-(2-hydroxypropylmethacrylamide).
(iv) the vinyl compounds of formula:
   CH₂═CH—R₉, CH₂═CH—CH₂—R₉ or CH₂═C(CH₃)—CH₂—R₉
wherein R₉ is chosen from a hydroxyl group, halogen atoms (such as Cl and F), NH₂, OR₁₄, wherein R₁₄ is chosen from a phenyl group and C₁ to C₁₂ alkyl groups (the monomer is a vinyl or allylic ether); acetamide (NHCOCH₃); a group OCOR₁₅, wherein R₁₅ is chosen from linear and branched alkyl groups comprising from 2 to 12 carbons (the monomer is a vinyl or allylic ester); and a group chosen from:
   linear and branched alkyl groups comprising from 1 to 18 carbon atoms, in which is optionally intercalated at least one hetero atom chosen from O, N, S and P; wherein the alkyl groups may also optionally be substituted with at least one substituent chosen from a hydroxyl group, halogen atoms (Cl, Br, I and F), and groups Si(R₄R₅), wherein R₄ and R₅, which may be identical or different, are each chosen from C₁ to C₆ alkyl groups and a phenyl group;
   C₃ to C₁₂ cycloalkyl groups such as isobornyl and cyclohexane,
   C₃ to C₂₀ aryl groups such as phenyl,
   C₄ to C₃₀ aralkyl groups (C₁ to C₈ alkyl groups) such as 2-phenylethyl and benzyl,
   4- to 12-membered heterocyclic groups comprising at least one hetero atom chosen from O, N and S, the ring being aromatic or non-aromatic,
   heterocycloalkyl groups (C₁ to C₄ alkyl groups), such as furfurylmethyl and tetrahydrofurfurylmethyl,
   wherein at least one of the cycloalkyl, aryl, aralkyl, heterocyclic and heterocycloalkyl groups may optionally be substituted with at least one substituent chosen from a hydroxyl group, halogen atoms, and linear and branched C₁ to C₄ alkyl groups in which are optionally intercalated at least one hetero atom chosen from O, N, S and P, wherein the alkyl groups may also be optionally substituted with at least one substituent chosen from a hydroxyl group, halogen atoms (Cl, Br, I and F), and groups Si(R₄R₅) wherein R₄ and R₅, which may be identical or different, are each chosen from C₁ to C₆ alkyl groups, and a phenyl group.

Examples of the vinyl monomers include vinyl cyclohexane and styrene. Examples of vinyl esters include vinyl acetate, vinyl propionate, vinyl butyrate, vinyl ethylhexanoate, vinyl neononanoate and vinyl neododecanoate.

Among the vinyl ethers that may be mentioned, examples include methyl vinyl ether, ethyl vinyl ether and isobutyl vinyl ether.
(v) (meth)acrylic, (meth)acrylamide and vinyl monomers comprising at least one group chosen from fluoro and perfluoro groups, such as ethylperfluorooctyl and 2-ethylperfluorohexyl (meth)acrylates;
(vi) silicone-based (meth)acrylic, (meth)acrylamide and vinyl monomers, such as methacryloxypropyltris(trimethylsiloxy)silane and acryloxypropylpolydimethylsiloxane;
(vii) ethylenically unsaturated monomers comprising at least one functional group chosen from carboxylic, phosphoric and sulfonic acid and anhydride functional groups, for instance acrylic acid, methacrylic acid, crotonic acid, maleic anhydride, itaconic acid, fumaric acid, maleic acid, acrylamidopropanesulfonic acid, vinylbenzoic acid and vinylphosphoric acid, and the salts thereof;
(viii) ethylenically unsaturated monomers comprising at least one tertiary amine functional group, for instance 2-vinylpyridine, 4-vinylpyridine, dimethylaminoethyl methacrylate, diethylaminoethyl methacrylate and dimethylaminopropylmethacrylamide, and the salts thereof.

The salts may be formed by neutralization of the anionic groups with a mineral base, such as LiOH, NaOH, KOH, Ca(OH)₂, NH₄OH or Zn(OH)₂; or with an organic base such as a primary, secondary or tertiary alkylamine, for example, triethylamine or butylamine. This primary, secondary or tertiary alkylamine may comprise at least one atom chosen from nitrogen and oxygen atoms and may thus comprise, for example, at least one alcohol functional group; mention may be made, for example, of amino-2-methyl-2-propanol, triethanolamine and dimethylamino-2-propanol. Mention may also be made, for example, of lysine and 3-(dimethylamino)propylamine.

Mention may further be made of the salts of mineral acids, such as sulfuric acid, hydrochloric acid, hydrobromic acid, hydriodic acid, phosphoric acid or boric acid. Mention may even further be made of the salts of organic acids, which may comprise at least one group chosen from carboxylic, sulfonic and phosphonic acid groups. They may be linear, branched, or cyclic aliphatic acids, or alternatively aromatic acids. These acids may also comprise at least one hetero atom chosen from O and N, for example, in the form of hydroxyl groups. Mention may be made, for example, of propionic acid, acetic acid, terephthalic acid, citric acid and tartaric acid.

The additional comonomers are, for example, chosen, alone or as a mixture, from C₁-C₁₈ alkyl and C₃-C₁₂ cycloalkyl (meth)acrylates, such as methyl acrylate, methyl methacrylate, isobornyl acrylate, isobornyl methacrylate, isobutyl acrylate, isobutyl methacrylate, 2-ethylhexyl acrylate, 2-ethylhexyl methacrylate, dodecyl acrylate, dodecyl methacrylate, stearyl acrylate, stearyl methacrylate, trifluoroethyl acrylate and trifluoroethyl methacrylate.

Mention may also be made, for example, of acrylic acid, methacrylic acid, methacryloxypropyltris(trimethylsiloxy)silane, acryloxypropyltris(trimethylsiloxy)silane, acryloxypropylpolydimethylsiloxane and methacryloxypropylpolydimethylsiloxane.

Among the additional monomers that may be present in the block comprising the at least one monomer with an optical effect of formula (I), and/or that may be present in the at least one other block not comprising a monomer with an optical effect of formula (I), mention may be made, for example, of the monomers with an optical effect of formulae (A), (B) and (C): 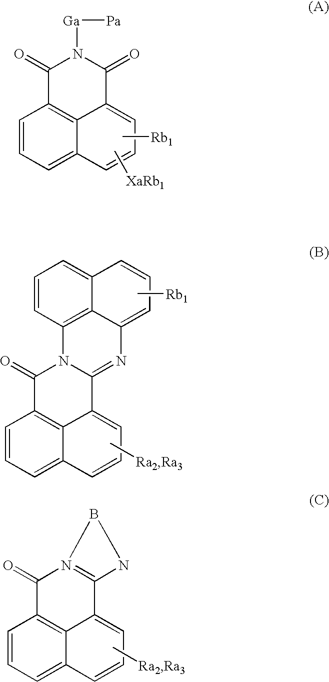
wherein:
Rb₁ is chosen from (i) a hydrogen atom, (ii) halogen atoms, (iii) linear, branched and cyclic, saturated and unsaturated carbon-based radicals comprising from 1 to 12 carbon atoms, optionally substituted with at least one group chosen from ═O, OH and NH₂ and/or optionally interrupted with at least one hetero atom chosen from O, N, P, Si and S; and (iv) a group NRR′ wherein R and R′, which may be identical or different, are each chosen from a hydrogen atom and linear, cyclic and branched, saturated C₁₋₆ hydrocarbon-based radicals, such as methyl, ethyl, propyl, isopropyl, n-butyl, isobutyl, tert-butyl, pentyl and hexyl radicals;
Ra₂ and Ra₃, which may be identical or different and are present on the same ring or each on a different ring, are each chosen from a hydrogen atom, halogen atoms, and a group of formula -Xa-Ga-Pa (II), with a proviso that at least one of the radicals Ra₂ and Ra₃ is a group of formula (II), wherein:
   Xa is chosen from groups —O—, —S—, —SO—, —SO₂—, —NH— and —NR₄— wherein R₄ is chosen from linear, branched and cyclic, saturated and unsaturated carbon-based radicals comprising from 1 to 30 carbon atoms, optionally substituted with at least one substituent chosen from ═O, OH, NH₂ and halogen atoms; and/or optionally interrupted with at least one hetero atom chosen from O, N, P, Si and S;
   Ga is chosen from linear, branched and cyclic, saturated and unsaturated divalent carbon-based radicals comprising from 1 to 32 carbon atoms, optionally substituted with at least one substituent chosen from ═O, OH, NH₂ and halogen atoms; and/or optionally interrupted with at least one hetero atom chosen from O, N, P, Si and S;
   Pa is a polymerizable group chosen from one of the following formulae: 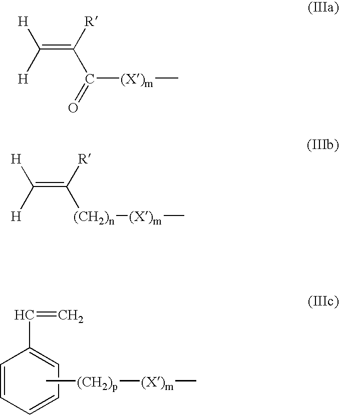
   wherein:
      R′ is chosen from H and linear and branched, saturated C₁₋₆ hydrocarbon-based radicals,
      X′ is chosen from O, NH and NR″ wherein R″ is a radical chosen from C₁₋₆ alkyl, C₆₋₁₀ aryl, (C₆₋₁₀)aryl(C₁₋₆)alkyl and (C₁₋₆)alkyl(C₆₋₁₀)aryl radicals, wherein at least one of the alkyl and aryl groups may be substituted with at least one substituent chosen from OH, halogen atoms, C₁₋₆ alkoxy and C₆₋₁₀ aryloxy groups; and
      m is equal to 0 or 1; n is equal to 0 or 1; and p is equal to 0, 1 or 2;
B is a divalent aromatic group chosen from the divalent aromatic groups of formulae (IVa) to (IVd) below: 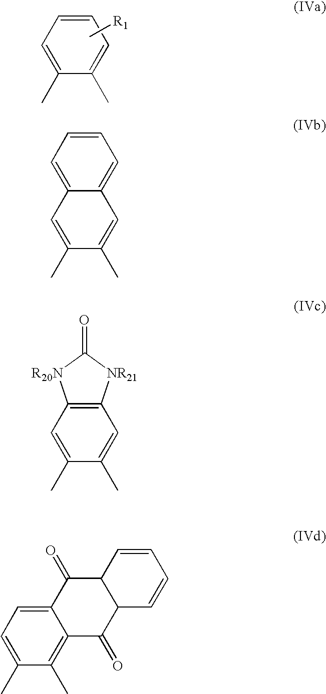
wherein:
   R₁ is chosen from linear, branched and cyclic, saturated and unsaturated carbon-based radicals comprising from 1 to 32 carbon atoms, optionally substituted with at least one substituent chosen from ═O, OH, NH₂ and halogen atoms; and
   R₂₀ and R₂₁, which may be identical or different, are each chosen from a hydrogen atom, linear and branched C₁₋₈ alkyl radicals and cyclopentyl, cyclohexyl, cyclooctyl, cyclodecyl, cyclododecyl, benzyl, naphthyl and phenyl radicals.

For example, the at least one monomer with an optical effect of formula (I) may be present in an amount ranging from 0.01% to 100% by weight, such as from 0.1% to 99.99% by weight, further such as from 0.5% to 70% by weight, even further such as from 1% to 40% by weight, and yet even further such as from 1.5% to 30% by weight, relative to the weight of the block comprising it.

Further, for example, the at least one monomer with an optical effect of formula (I) may be present in an amount ranging from 0.01% to 70% by weight, such as from 0.1% to 50% by weight, further such as from 0.5% to 30% by weight, and even further such as from 1% to 20% by weight, relative to the total weight of the polymer.

Each of the blocks of the polymer disclosed herein may comprise one or more monomers of formula (I), which may be identical or different depending on the block.

The at least one additional monomer may be present in an amount ranging from 0 to 99.99% by weight, such as from 0.01% to 99.9% by weight, further such as from 30% to 99.5% by weight, even further such as from 60% to 99% by weight, for example, from 70% to 98.5% by weight, relative to the weight of the block comprising the at least one additional monomer and the at least one monomer with an optical effect of formula (I). The at least one additional monomer may be present in an amount of 100% by weight in possible block(s) not comprising any monomer of formula (I).

The at least one additional monomer may be present in an amount ranging from 30% to 99.99% by weight, such as from 50% to 99.9% by weight, further such as from 70% to 99.5% by weight, and even further such as from 80% to 99% by weight, relative to the total weight of the polymer.

For example, the intermediate block (or segment) comprises at least one constituent monomer m1 of the first block which may be chosen from the additional monomers, and at least one constituent monomer m2 of the second block chosen from the additional monomers other than the monomer m1.

In one embodiment, the polymer may comprise at least one first block that comprises from 0.5% to 15% by weight such as from 1% to 10% by weight of the at least one monomer of formula (I) and from 85% to 99.5% by weight such as from 90% to 99% by weight of the at least one additional monomer, wherein the percentages are given relative to the total weight of the block. The at least one second block may comprise, for example, 100% by weight of the at least one additional monomer. The intermediate block (or segment) may comprise, for example, at least one constituent monomer m1 of the first block chosen from the additional monomers, and at least one constituent monomer m2 of the second block chosen from the additional monomers other than the monomer m1.

For example, in the polymer disclosed herein, the at least one first block may be chosen from:
a) a block with a Tg of greater than or equal to 40° C.,
b) a block with a Tg of less than or equal to 20° C.,
c) a block with a Tg of between 20 and 40° C.,
and the at least one second block may be chosen from categories a), b) and c) but different from the first block.

The block comprising the at least one monomer with an optical effect of formula (I) may have, for example, a Tg of greater than or equal to 40° C. such as greater than or equal to 60° C.

In this case, the other block(s), if they do not comprise any monomers with an optical effect of formula (I), may have a Tg of less than or equal to 40° C. such as less than or equal to 20° C.

If the at least one second block comprises at least one monomer with an optical effect of formula (I), it may, for example, have a Tg of less than or equal to 40° C. such as less than or equal to 20° C.

When the polymer comprises a block with a Tg of greater than or equal to 40° C., this block may have, for example, a Tg ranging from 40° C. to 150° C., such as from 50° C. to 120° C. and further such as from 60° C. to 120° C.

The polymer may then, for example, comprise, in total or in part, monomers whose homopolymers have a Tg in the desired range, such as greater than or equal to 40° C. It may also comprise monomers with a Tg outside this range. These monomers and their concentrations are chosen in an appropriate manner by a person skilled in the art, for example, on the basis of Fox's law, to obtain a block of desired Tg.

Among these monomers, mention may be made, for example, of:
the methacrylates of formula: CH₂═C(CH₃)—COOR₁
wherein R₁ is chosen from linear and branched unsubstituted alkyl groups comprising from 1 to 4 carbon atoms, such as a methyl, ethyl, propyl and isobutyl groups, or R₁ is chosen from C₄ to C₁₂ cycloalkyl groups, such as an isobornyl group;
the acrylates of formula: CH₂═CH—COOR₂
wherein R₂ is chosen from a tert-butyl group and C₄ to C₁₂ cycloalkyl groups such as an isobornyl group;
the (meth)acrylamides of formula: CH₂═CR′—CO—NR₇R₈
wherein R′ is H or CH₃, and R₇ and R₈, which may be identical or different, are each chosen from a hydrogen atom and linear and branched C₁ to C₁₂ alkyl groups, such as an n-butyl, t-butyl, isopropyl, isohexyl, isooctyl and isononyl groups; or alternatively R₇ is H and R₈ is a 1,1-dimethyl-3-oxobutyl group,
and mixtures thereof.

Among the monomers whose homopolymers have a glass transition temperature Tg of greater than or equal to 40° C., mention may be made, for example, of methyl methacrylate, ethyl methacrylate, isobutyl methacrylate, tert-butyl (meth)acrylate, (meth)acrylic acid, isobornyl (meth)acrylate, N-butylacrylamide, N-t-butylacrylamide, N-isopropylacrylamide, N,N-dimethylacrylamide and N,N-dibutylacrylamide, and mixtures thereof.

When the polymer comprises a block with a Tg of less than or equal to 20° C., this block may have, for example, a Tg ranging from −100° C. to 20° C., such as from −80° C. to 15° C. and further such as from −50° C. to 0° C.

The polymer may then, for example, comprise, in total or in part, monomers whose homopolymers have a Tg in the desired range, such as less than or equal to 20° C. It may also comprise monomers with a Tg outside this range. These monomers and their concentrations are chosen in an appropriate manner by a person skilled in the art, for example, on the basis of Fox's law, to obtain a block of desired Tg.

Among these monomers, mention may be made, for example, of:
the acrylates of formula CH₂═CHCOOR₃,
wherein R₃ is chosen from linear and branched, unsubstituted C₁ to C₁₂ alkyl groups, with the exception of the tert-butyl group, in which is optionally intercalated at least one hetero atom chosen from O, N and S,
the methacrylates of formula CH₂═C(CH₃)—COOR₄,
wherein R₄ is chosen from linear and branched, unsubstituted C₆ to C₁₂ alkyl groups, in which is optionally intercalated at least one hetero atom chosen from O, N and S;
the vinyl esters of formula R₅—CO—O—CH═CH₂
wherein R₅ is chosen from linear and branched C₄ to C₁₂ alkyl groups;
C₄-C₁₂ alkyl vinyl ethers, such as methyl vinyl ether and ethyl vinyl ether;
N—(C₄ to C₁₂ alkyl) acrylamides such as N-octylacrylamide,
and mixtures thereof.

Among these monomers, mention may be made, for example, of methyl acrylate, ethyl acrylate, isobutyl acrylate and 2-ethylhexyl (meth)acrylate, and mixtures thereof.

When the polymer comprises a block with a Tg of between 20° C. and 40° C., it may, for example, comprise, in total or in part, monomers whose homopolymers have a Tg in the desired range, and among which mention may be made, for example, of n-butyl methacrylate, cyclohexyl acrylate, cyclododecyl acrylate, neopentyl acrylate and isodecylacrylamide, and mixtures thereof.

In one embodiment, the polymer disclosed herein comprises in at least one block, such as in each of the blocks, at least one monomer chosen from (meth)acrylic acid esters; it may optionally also comprise at least one second monomer chosen from acrylic acid and methacrylic acid, and mixtures thereof.

For example, all the monomers other than the monomers with optical effects of formula (I) are chosen from (meth)acrylic acid esters and (meth)acrylic acid.

According to a first embodiment, the polymer disclosed herein comprises at least one first block with a Tg of greater than or equal to 40° C., such as greater than or equal to 50° C., and further such as greater than or equal to 60° C., and at least one second block with a Tg of less than or equal to 20° C., such as less than or equal to 10° C. and further such as less than or equal to 0° C.

For example, the block with a Tg of greater than or equal to 40° C. may be present in an amount ranging from 20% to 95% by weight, such as from 30% to 80% by weight, and further such as from 50% to 75% by weight, relative to the weight of the final polymer.

For example, the block with a Tg of less than or equal to 20° C. may be present in an amount ranging from 5% to 80% by weight, such as from 15% to 50% by weight, and further such as from 25% to 45% by weight, relative to the weight of the final polymer.

The weight-average mass (Mw) of the polymer as disclosed herein is, for example, less than or equal to 300 000, ranging, for example, from 35 000 to 200 000 such as from 40 000 to 150 000.

The number-average mass (Mn) of the polymer as disclosed herein is, for example, less than or equal to 70 000, ranging, for example, from 5000 to 60 000 such as from 6000 to 50 000.

The weight-average (Mw) and number-average (Mn) molar masses are determined by gel permeation liquid chromatography (THF solvent, calibration curve established with linear polystyrene standards, refractometric and UV detector).

For example, the polydispersity index (Ip) of the polymer as disclosed herein is greater than 2, ranging, for example, from 2 to 9; such as greater than or equal to 2.5, ranging, for example, from 2.5 to 8; and further such as greater than or equal to 2.8, ranging, for example, from 2.8 to 7. The polydispersity index (Ip) of the polymer is equal to the ratio of the weight-average mass Mw to the number-average mass Mn.

The polymer as disclosed herein has an absorption wavelength ranging, for example, from 200 to 550 nm such as from 220 to 500 nm, and further such as from 240 to 490 nm.

The polymer as disclosed herein has an emission wavelength ranging, for example, from 350 to 700 nm, such as from 390 to 650 nm, further such as from 400 to 600 nm.

The polymer as disclosed herein may be obtained by solution free-radical polymerization according to the following preparation process:
a portion of the polymerization solvent may be introduced into a suitable reactor, and the system is heated until the appropriate temperature for the polymerization (ranging, for example, from 60 to 120° C.) is reached,
once this temperature has been reached, the constituent monomers of the first block may be added, in the presence of some of the polymerization initiator,
after a time T corresponding to a maximum degree of conversion, for example, of 90%, the constituent monomers of the second block and the rest of the initiator may be introduced,
the mixture is left to react for a time T′ (ranging, for example, from 3 to 6 hours), at the end of which the mixture is cooled to room temperature (25° C.) so as to obtain the polymer dissolved in the polymerization solvent.

The term “polymerization solvent” means a solvent or a solvent mixture chosen, for example, from ethyl acetate, butyl acetate, C₁-C₆ alcohols such as isopropanol or ethanol, aliphatic alkanes such as isododecane, and mixtures thereof. For example, the polymerization solvent is a mixture of butyl acetate and isopropanol. In one embodiment, the polymerisation solvent is isododecane.

For example, the polymer disclosed herein is not water-soluble, i.e., the polymer is not soluble in water or in a mixture of water and at least one monoalcohol chosen from linear and branched monoalcohols comprising from 2 to 5 carbon atoms, such as ethanol, isopropanol and n-propanol, without a pH modification, at an active material content of at least 1% by weight, at room temperature (25° C.).

The present disclosure also relates to compositions, such as cosmetic compositions, comprising at least one polymer of the structure as described above, in a physiologically acceptable such as a cosmetically acceptable medium.

The polymers disclosed herein may be present, alone or as a mixture, in the compositions disclosed herein in an amount ranging, for example, from 0.01% to 75% by weight, such as from 0.1% to 70% by weight, such as from 1% to 65% by weight, further such as from 3% to 60% by weight, even further such as from 5% to 50% by weight and yet even further such as from 6% to 25% by weight, relative to the total weight of the composition.

The polymers disclosed herein may be present in the composition in dissolved form, for example, in water, in an oil or in an organic solvent, or alternatively in the form of an aqueous or organic dispersion.

In one embodiment, the polymers disclosed herein are soluble or dispersible in at least one of the phases of the composition comprising them.

The cosmetic or pharmaceutical compositions disclosed herein further comprise, a physiologically acceptable medium, such as a cosmetically, dermatologically or pharmaceutically acceptable medium, i.e., a medium that is compatible with keratin materials such as facial or bodily skin, the hair, the eyelashes, the eyebrows and the nails.

The composition may comprise a hydrophilic medium comprising water or a mixture of water and at least one solvent chosen from hydrophilic organic solvents, for instance, alcohols such as linear or branched lower monoalcohols comprising from 2 to 5 carbon atoms, for instance ethanol, isopropanol or n-propanol, and polyols, for instance glycerol, diglycerol, propylene glycol, sorbitol or pentylene glycol, and polyethylene glycols, or alternatively hydrophilic C₂ ethers and C₂-C₄ aldehydes.

The water or the mixture of water and at least one hydrophilic organic solvent may be present in an amount ranging, for example, from 0.1% to 99% by weight, such as from 10% to 80% by weight, relative to the total weight of the composition.

The composition may also be anhydrous.

The composition may also comprise a fatty phase which may comprise at least one fatty substance chosen from fatty substances that are liquid at room temperature (in general 25° C.) and fatty substances that are solid at room temperature, such as waxes, pasty fatty substances and gums, and mixtures thereof. These fatty substances may be of animal, plant, mineral or synthetic origin. This fatty phase may also comprise at least one lipophilic organic solvent.

As fatty substances that are liquid at room temperature, often referred to as oils, which may be used herein, mention may be made, for example, of hydrocarbon-based oils of animal origin such as perhydrosqualene; hydrocarbon-based plant oils such as liquid triglycerides of fatty acids comprising from 4 to 10 carbon atoms, for instance heptanoic or octanoic acid triglycerides, or alternatively sunflower oil, maize oil, soybean oil, grapeseed oil, sesame seed oil, apricot oil, macadamia oil, castor oil, avocado oil, caprylic/capric acid triglycerides, jojoba oil, shea butter, linear or branched hydrocarbons of mineral or synthetic origin, such as liquid paraffin and derivatives thereof, petroleum jelly, polydecenes, hydrogenated polyisobutene such as parleam; synthetic esters and ethers, such as synthetic esters and ethers of fatty acids, for instance purcellin oil, isopropyl myristate, 2-ethylhexyl palmitate, 2-octyldodecyl stearate, 2-octyldodecyl erucate, isostearyl isostearate; hydroxylated esters, for instance isostearyl lactate, octyl hydroxystearate, octyldodecyl hydroxystearate, diisostearyl malate, triisocetyl citrate, and fatty alcohol heptanoates, octanoates and decanoates; polyol esters, for instance propylene glycol dioctanoate, neopentyl glycol diheptanoate and diethylene glycol diisononanoate; and pentaerythritol esters; fatty alcohols comprising from 12 to 26 carbon atoms, for instance octyldodecanol, 2-butyloctanol, 2-hexyldecanol, 2-undecylpentadecanol and oleyl alcohol; partially hydrocarbon-based fluoro oils and/or partially silicone-based fluoro oils; silicone oils, for instance volatile or non-volatile, linear or cyclic polymethylsiloxanes (PDMSs), which are liquid or pasty at room temperature, for instance cyclomethicones, dimethicones, optionally comprising a phenyl group, for instance phenyl trimethicones, phenyltrimethylsiloxydiphenylsiloxanes, diphenylmethyldimethyltrisiloxanes, diphenyl dimethicones, phenyl dimethicones and polymethylphenylsiloxanes; mixtures thereof.

These oils may be present in an amount ranging, for example, from 0.01% to 90% by weight, such as from 0.1% to 85% by weight, relative to the total weight of the composition.

The composition disclosed herein may also comprise at least one physiologically acceptable organic solvent.

The at least one physiologically acceptable organic solvent may be generally present in an amount ranging from 0.1% to 90% by weight, such as from 0.5% to 85% by weight, further such as from 10% to 80% by weight, and even further such as from 30% to 50% by weight, relative to the total weight of the composition.

Mention may be made, for example, besides the hydrophilic organic solvents mentioned above, of ketones that are liquid at room temperature such as methyl ethyl ketone, methyl isobutyl ketone, diisobutyl ketone, isophorone, cyclohexanone and acetone; propylene glycol ethers that are liquid at room temperature, such as propylene glycol monomethyl ether, propylene glycol monomethyl ether acetate, and dipropylene glycol mono-n-butyl ether; short-chain esters (comprising from 3 to 8 carbon atoms in total), such as ethyl acetate, methyl acetate, propyl acetate, n-butyl acetate and isopentyl acetate; ethers that are liquid at 25° C., such as diethyl ether, dimethyl ether or dichlorodiethyl ether; alkanes that are liquid at 25° C., such as decane, heptane, dodecane, isododecane and cyclohexane; aromatic cyclic compounds that are liquid at 25° C., such as toluene and xylene; aldehydes that are liquid at 25° C., such as benzaldehyde and acetaldehyde, and mixtures thereof.

As used herein, the term “wax” means a lipophilic compound that is solid at room temperature (25° C.), which undergoes a reversible solid/liquid change of state, and has a melting point of greater than or equal to 25° C., such as up to 120° C. By bringing the wax to the liquid state (melting), it is possible to make it miscible with the oils possibly present and to form a microscopically homogeneous mixture, but, on returning the temperature of the mixture to room temperature, recrystallization of the wax is obtained in the oils of the mixture. The melting point of the wax may be measured using a differential scanning calorimeter (DSC), for example, the calorimeter sold under the name DSC 30 by the company Mettler.

The waxes may be chosen from hydrocarbon-based waxes, fluoro waxes and silicone waxes and may be of plant, mineral, animal and/or synthetic origin. For example, the waxes have a melting point of greater than 30° C. such as greater than 45° C. As the waxes that may be used in the composition disclosed herein, mention may be made, for example, of beeswax, carnauba wax or candellila wax, paraffin, microcrystalline waxes, ceresin or ozokerite, synthetic waxes, for instance polyethylene waxes or Fischer-Tropsch waxes, and silicone waxes, for instance alkyl or alkoxy dimethicones comprising from 16 to 45 carbon atoms.

The gums are generally polydimethylsiloxanes (PDMSs) of high molecular weight or cellulose gums or polysaccharides, and the pasty substances are generally hydrocarbon-based compounds, for instance lanolins and derivatives thereof, or PDMSs.

The nature and amount of the solid substances in the composition disclosed herein depend on the desired mechanical properties and textures. For example, the composition may comprise from 0.1% to 50% by weight, such as from 1% to 30% by weight, of waxes relative to the total weight of the composition.

The composition disclosed herein may also comprise, in a particulate phase, at least one additive chosen from pigments, nacres, and fillers usually used in cosmetic compositions.

The composition may also comprise other dyestuffs chosen from water-soluble dyes and liposoluble dyes that are well known to those skilled in the art.

The term “pigments” means white or colored, mineral or organic particles of any shape, which are insoluble in the physiological medium and are intended to color the composition.

The term “fillers” means colorless or white, mineral or synthetic, lamellar or non-lamellar particles intended to give body or rigidity to the composition, and/or softness, a matt effect and uniformity to the makeup result.

The term “nacres” means iridescent particles of any form, produced, for example, by certain molluscs in their shell, or synthesized.

The pigments may be present in the composition in an amount ranging, for example, from 0.01% to 25% by weight, such as from 3% to 10% by weight, relative to the total weight of the composition. They may be white or colored, and mineral or organic. Mention may be made, for example, of titanium oxide, zirconium oxide, cerium oxide, zinc oxide, iron oxide or chromium oxide, ferric blue, chromium hydrate, carbon black, ultramarines (aluminosilicate polysulfides), manganese pyrophosphate and certain metallic powders such as silver or aluminium powder. Mention may also be made, for example, of the D&C pigments and lakes commonly used to give the lips and the skin a makeup effect, which include, for example, calcium, barium, aluminium, strontium or zirconium salts.

The nacres may be present in the composition in an amount ranging, for example, from 0.01% to 20% by weight, such as from 3% to 10% by weight, relative to the total weight of the composition. Among the nacres that may be used herein, mention may be made, for example, of natural mother-of-pearl, mica coated with titanium oxide, with iron oxide, with natural pigment or with bismuth oxychloride, and also colored titanium mica.

The liposoluble or water-soluble dyes may be present in the composition, alone or as a mixture, in an amount ranging, for example, from 0.001% to 15% by weight, such as from 0.01% to 5% by weight, further such as from 0.1% to 2% by weight, relative to the total weight of the composition. Among the liposoluble or water-soluble dyes that may be used herein, mention may be made, for example, of the disodium salt of ponceau, the disodium salt of alizarin green, quinoline yellow, the trisodium salt of amaranth, the disodium salt of tartrazine, the monosodium salt of rhodamine, the disodium salt of fuchsin, xanthophyll, methylene blue, cochineal carmine, halo-acid dyes, azo dyes, anthraquinone dyes, copper sulfate, iron sulfate, Sudan brown, Sudan red and annatto, and also beetroot juice and carotene.

The composition as disclosed herein may also comprise at least one filler, in an amount ranging, for example, from 0.01% to 50% by weight such as from 0.02% to 30% by weight, relative to the total weight of the composition. The fillers may be mineral or organic in any form, platelet-shaped, spherical or oblong. Mention may be made, for example, of talc, mica, silica, kaolin, polyamide (Nylon®) powders, poly-β-alanine powder and polyethylene powder, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie) or acrylic acid copolymers (Polytrap® from the company Dow Corning) and silicone resin microbeads (for example, Tospearls® from Toshiba), elastomeric polyorganosiloxane particles, precipitated calcium carbonate, magnesium carbonate, magnesium hydrocarbonate, hydroxyapatite, hollow silica microspheres (Silica Beads® from Maprecos), glass or ceramic microcapsules, and metal soaps derived from organic carboxylic acids comprising from 8 to 22 carbon atoms such as from 12 to 18 carbon atoms, for example, zinc, magnesium or lithium stearate, zinc laurate or magnesium myristate.

The composition may also comprise at least one additional polymer such as at least one film-forming polymer. As used herein, the term “film-forming polymer” means a polymer capable, by itself or in the presence of an auxiliary film-forming agent, of forming a continuous film that adheres to a support such as a keratin material. Among the film-forming polymers that may be used in the composition disclosed herein, mention may be made, for example, of synthetic polymers, of free-radical type or of polycondensate type, polymers of natural origin and mixtures thereof, such as acrylic polymers, polyurethanes, polyesters, polyamides, polyureas and cellulose-based polymers, for instance nitrocellulose.

The composition as disclosed herein may also comprise at least one ingredient chosen from ingredients commonly used in cosmetics, such as vitamins, thickeners, gelling agents trace elements, softeners, sequestering agents, fragrances, acidifying or basifying agents, preserving agents, sunscreens, surfactants, antioxidants, agents for preventing hair loss, antidandruff agents, propellants and ceramides or mixtures thereof.

A person skilled in the art will take care to select this or these optional additional compound(s), and/or the amount thereof, such that the advantageous properties of the composition disclosed herein are not, or are not substantially, adversely affected by the envisaged addition.

The composition disclosed herein may be in the form of a suspension, a dispersion, such as a dispersion of oil in water via vesicles; an optionally thickened or even gelled aqueous or oily solution; an oil-in-water, water-in-oil or multiple emulsion; a gel or a mousse; an oily or emulsified gel; a dispersion of vesicles, such as a dispersion of lipid vesicles; a two-phase or multiphase lotion; a spray; a free, compact or cast powder; or an anhydrous paste. This composition may have the appearance of a lotion, a cream, a salve, a soft paste, an ointment, a mousse, a cast or moulded solid, such as in stick or dish form, and/or a compacted solid.

A person skilled in the art will be able to choose the appropriate galenical form, and also the method for preparing it, on the basis of his or her general knowledge, taking into account, for example, the nature of the constituents used, such as their solubility in the support, and also the intended application of the composition.

The cosmetic composition as disclosed herein may be in the form of a care and/or makeup product for bodily or facial skin, the lips, the nails, the eyelashes, the eyebrows and/or the hair, an antisun product, a self-tanning product, and/or a hair product for caring for, treating, shaping, making up or coloring the hair.

It may also be in the form of a makeup composition, for example, a complexion product such as a foundation, a makeup rouge or an eyeshadow; a lip product such as a lipstick or a lipcare product; a concealer product; a blusher, a mascara or an eyeliner; an eyebrow makeup product, a lip pencil or an eye pencil; a nail product such as a nail varnish or a nailcare product; a body makeup product; and/or a hair makeup product (hair mascara or hair lacquer).

The composition may also be in the form of a protective or care composition for the skin of the face, the neck, the hands or the body, such as an anti-wrinkle composition, a moisturizing or treating composition; an antisun composition and/or an artificial tanning composition.

The composition may also be in the form of a hair product, such as for coloring, holding the hairstyle, shaping the hair, caring for, treating or cleansing the hair, such as shampoos, hairsetting gels or lotions, blow-drying lotions, and fixing and styling compositions such as lacquers or sprays.

In one embodiment, the composition may be in the form of a nail varnish that may comprise, besides the at least one polymer disclosed herein, at least one organic solvent, at least one film-forming polymer and optionally at least one pigment and/or at least one dye.

In another embodiment, the composition may be in the form of a foundation that may comprise, besides the at least one polymer disclosed herein, at least one oil in a fatty phase, at least one pigment and optionally an aqueous phase.

In another embodiment, the composition may be in the form of an anti-ageing or anti-wrinkle composition, for example, intended to be applied to the face and/or the neck, such as to the wrinkled areas of the face, for example, around the eyes.

It has been found surprisingly that the use of the at least one polymer disclosed herein makes it possible to obtain a composition that may be applied to the skin and may give an immediate tensioning effect on already-formed wrinkles and/or fine lines. For example, the polymers disclosed herein can be used as tensioning agents, since they make it possible to form an effective tensioning film with effective rigidity, while at the same time being supple so as to avoid an annoying tautness of keratin materials such as the skin, during the application of a composition comprising such agents.

In this case, the block polymer is, for example, non-elastomeric and water-insoluble. The term “water-insoluble polymer” means that the polymer is not soluble in water or in a mixture of water and at least one monoalcohol chosen from linear and branched C₂-C₅ monoalcohols, for instance ethanol, isopropanol or n-propanol, without pH modification, at an active material content of at least 1% by weight, at room temperature (25° C.).

In this embodiment, the first block has, for example, a Tg of greater than or equal to 85° C., such as from 90° C. to 150° C., and further such as from 100° C. to 120° C. The block with a Tg of greater than or equal to 85° C. is present in an amount ranging, for example, from 50% to 90% by weight, such as from 60% to 80% by weight relative to the weight of the final polymer. The second block has, for example, a Tg of less than or equal to 20° C., such as from −100° C. to 20° C., further such as from −80° C. to 15° C. and even further such as from −70° C. to 10° C. The block with a Tg of less than or equal to 20° C. is present in an amount ranging, for example, from 5% to 50% by weight, such as from 10% to 40% by weight, relative to the weight of the final polymer.

The composition disclosed herein may also comprise at least one anti-ageing active agent chosen, for example, from desquamating agents, moisturizers, agents for stimulating keratinocyte proliferation and/or differentiation, agents for stimulating collagen and/or elastin synthesis or for preventing their degradation, depigmenting agents, anti-glycation agents, agents for stimulating glycoaminoglycan synthesis, dermo-decontracting agents or muscle relaxants, antioxidants and free-radical scavengers, and mixtures thereof.

Further disclosed herein is the use of the polymers disclosed herein as tensioning agents in a cosmetic composition, such as in an anti-wrinkle composition.

Also disclosed herein is a cosmetic process for treating wrinkled skin, such as the contour of the eyes, comprising applying to the wrinkled skin a cosmetic composition comprising, in a cosmetically acceptable medium, at least one polymer as defined above.

Even further disclosed herein is a cosmetic treatment process, such as for making up or caring for keratin materials, such as bodily or facial skin, the lips, the nails, the eyelashes, the eyebrows and/or the hair, comprising applying to the keratin materials a cosmetic composition as defined above.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The examples that follow are given as non-limiting illustrations of the present disclosure.

Method for Measuring the Wavelength (Emission and Absorption)

The wavelength measurement is performed using a Varian Cary Eclipse fluorimeter.

Unless otherwise indicated, this measurement is performed in the following manner:
20 mg of a sample are placed in a 50 ml cylinder. To dissolve the sample, the cylinder is filled to 50 ml with a suitable solvent, for example, dichloromethane (DCM), chloroform, isododecane, heptane or dimethyl sulfoxide (DMSO). The resulting solution is mixed and 250 microlitres are taken and placed in a 50 ml cylinder, which is then filled to 50 ml again with the solvent.

The whole is mixed and a sample of the solution is taken and placed in a closed quartz cuvette 10 mm thick, which is then placed in the measuring chamber.

### EXAMPLE 1

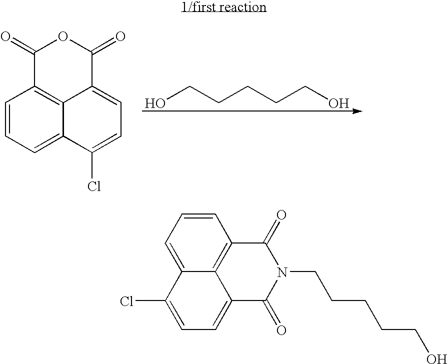

26.9 g (0.116 mol) of 4-chloro-1,8-naphthalic anhydride were placed in a 2 liter round-bottomed flask under an inert atmosphere (argon), and 450 ml of toluene were then added. The mixture was stirred at 500 rpm for a few minutes and 14.0 g (0.139 mol) of 1-hexylamine predissolved in 100 ml of toluene were introduced dropwise. The mixture was refluxed and a further 50 ml of toluene were then added. Refluxing was continued for 24 hours. The resulting reaction mixture was then allowed to cool to room temperature. The product was concentrated under reduced pressure. The organic phase was recovered and recrystallized in ethanol. 29.0 g of pale yellow crystals were obtained (79.4% yield).

Characterization

¹H NMR (CDCl₃, 400 MHz) δ: 8.67-8.65 (2H), 8.51-8.49 (1H), 7.87-7.85 (1H) 7.83-7.81 (1H), 4.18-4.14 (2H), 1.74-1.70 (2H), 1.44-1.32 (6H), 0.90-0.87 (3H).

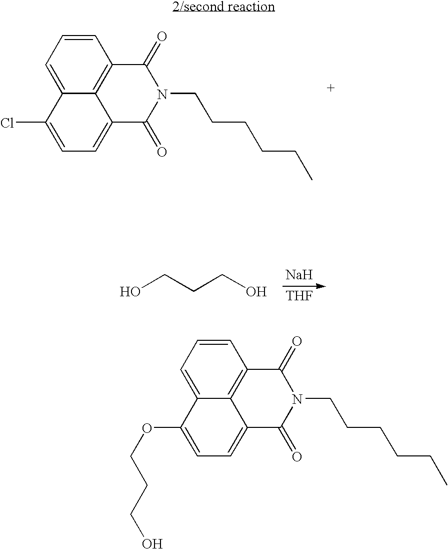

1.5 g (64 mmol) of sodium hydrate (NaH) were placed in a round-bottomed flask under an inert atmosphere of argon; 300 ml of THF (tetrahydrofuran) were added and the reaction mixture was cooled to 0° C. 2.4 g (32 mmol) of 1,3-propanediol premixed with 100 ml of THF were added and the mixture was stirred vigorously. The mixture was heated at 60° C. for 20 minutes and was then cooled to room temperature (25° C.). A mixture comprising 10 g (32 mmol) of naphthalimide in 150 ml of THF was then added. The mixture was refluxed for 3 hours, 10 ml of ethanol were then added, this mixture was heated for 15 minutes and the solvent was evaporated off under reduced pressure to give a greenish residue, which was washed with ethyl acetate and with water. 8.6 g of a pale yellow powder were thus obtained (75.5% yield).

Characterization

¹H NMR (CDCl₃, 400 MHz) δ: 8.61-8.59 (2H), 8.55-8.53 (1H), 7.72-7.68 (1H), 7.08-7.06 (1H), 4.45-4.42 (2H), 4.17-4.13 (2H), 4.00-3.97 (2H), 2.28-2.22 (2H), 1.76-1.70 (2H), 1.44-1.32 (6H), 0.90-0.87 (3H).

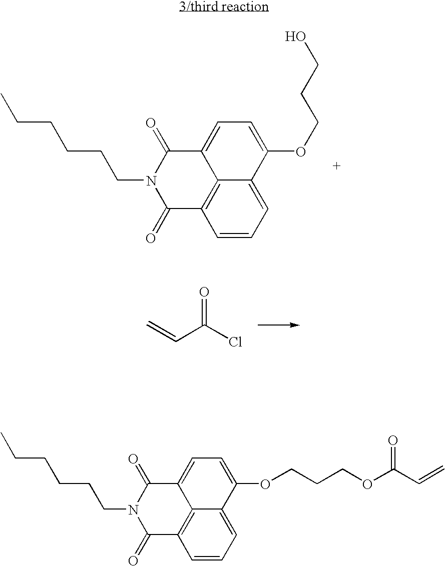

8.0 g (22.5 mmol) of 4-(oxy-3-propanol)-N-hexyl napthalimide were placed in a three-necked round-bottomed flask equipped with a condenser and placed under an inert atmosphere of argon. 150 ml of dichloromethane were added and the solution was stirred until a homogeneous solution was obtained. 4.6 g (45.0 mmol) of triethanolamine were then added, followed by addition of 4.1 g (45.0 mmol) of acryloyl chloride, with stirring at 0° C. The reaction progress was monitored by TLC (thin-layer chromatography), and when no more starting compounds remained, 50 ml of water were added. The reaction solution was then washed with saturated NaCl solution and then dried over sodium sulphate. The solvents were evaporated off under reduced pressure to give 9.2 g of a pale yellow powder (100% yield).

Characterization

¹H NMR (CDCl₃, 400 MHz) δ: 8.59-8.57 (1H), 8.54-8.52 (1H), 8.51-8.50 (1H), 7.70-7.66 (1H), 7.03-7.01 (1H), 6.44-6.40 (1H), 6.17-6.10 (1H), 5.86-5.83 (1H), 4.49-4.46 (2H), 4.39-4.36 (2H), 4.16-4.12 (2H), 2.38-2.35 (2H), 1.71-1.69 (2H), 1.44-1.31 (6H), 0.89-0.86 (3H).

### EXAMPLE 2

33 g of isododecane were placed in a 500 ml reactor and then heated to 90° C.

2 g of the optical-brightening monomer prepared according to Example 1 were dissolved in 10 ml of toluene, and 40 g of isobornyl acrylate, 28 g of isobutyl methacrylate, 37 g of isododecane and 0.6 g of initiator 2,5-bis(2-ethylhexanoylperoxy)-2,5-dimethylhexane (Trigonox® 141 from Akzo Nobel) were then added. This mixture was stirred for 1 hour in the reactor at 90° C. The whole was maintained at 90° C. for 1 hour 30 minutes.

30 g of 2-ethylhexyl acrylate, 30 g of isododecane and 0.4 g of 2,5-bis(2-ethylhexanoylperoxy)-2,5-dimethylhexane were then introduced into the above mixture, at 90° C. and over 30 minutes. The mixture was maintained at 90° C. for 4 hours and was then cooled to 25° C.

After replacing the toluene with isododecane, a solution comprising 50% of polymer solids in isododecane was obtained.

The polymer had a weight-average mass (Mw) of 48 500 and a number-average mass (Mn) of 12 300, i.e., a polydispersity index (Ip) of 3.9.

This polymer comprised a first block of isobornyl acrylate, isobutyl methacrylate and optical-brightening monomer as disclosed herein, and a 2-ethylhexyl acrylate second block, and also an intermediate segment.

### EXAMPLE 3

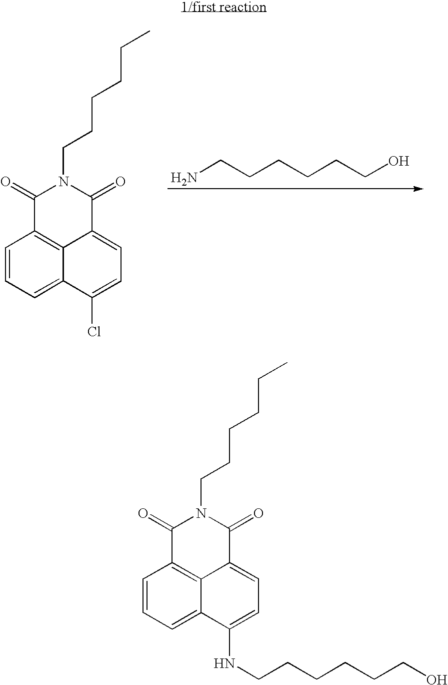

2.71 g (10.85 mmol) of CuSO₄, 5H₂O and 10.07 g (31.9 mmol) of 4-chloro-N-hexyl-1,8-napthalimide were mixed together in 170 ml of ethanol in a round-bottomed flask, under argon, and the reaction medium was then brought to reflux.

7.7 g (65.7 mmol) of aminohexan-6-ol were added to the reaction medium dropwise and stirring was then continued for 16 hours. The solution was then cooled to 25° C. and the product was precipitated by addition of 200 ml of water, and then filtered off on a sinter funnel. A solid yellow-green product was obtained, which was taken up in 100 ml of dichloromethane. This solution was washed with water and the aqueous phase was then evaporated to give an oil, which was filtered on silica (0.043-0.060 microns, eluting with dichloromethane and then with acetone).

1.6 g of a yellow oil were finally obtained (12.6% yield).

Characterization

¹H NMR (CDCl₃, 400 MHz) δ ppm: 0.87 (t, 3H, CH₃); 1.50 (m, 8H, CH₂); 1.81 (m, 2H, NCH₂CH₂); 3.40 (t, 2H, CH₂OH); 3.68 (t, 2H, NHCH₂); 4.14 (t, 2H, NCH₂); 5.26 (s, 1H, NH); 6.71 (d, 1H, ArH); 7.60 (t, 1H, ArH); 8.06 (d, 1H, ArH); 8.43 (d, 1H, ArH); 8.57 (d, 1H, ArH).

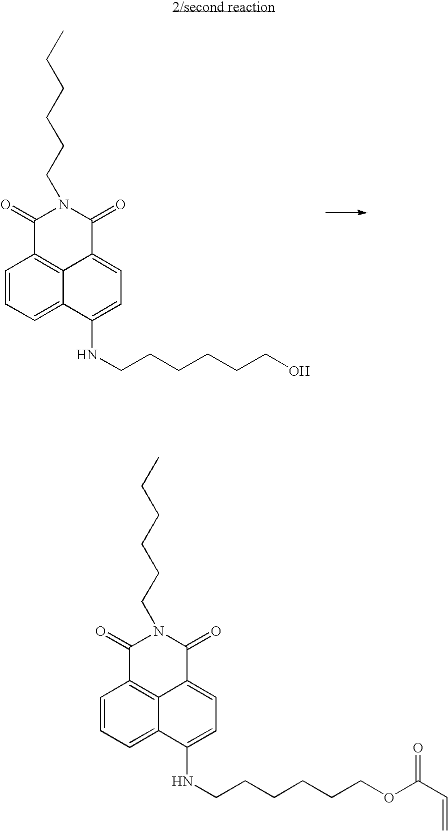

1.5 g (3.9 mmol) of 4-(amino-6-hexanol)-N-hexyl naphthalimide were placed in a three-necked round-bottomed flask equipped with a condenser and placed under an inert atmosphere of argon. 50 ml of dichloromethane were added and the solution was stirred until a homogeneous solution was obtained. 0.8 g (7.9 mmol) of triethanolamine were then added, followed by addition of 0.7 g (7.9 mmol) of acryloyl chloride, with stirring at 0° C. The temperature was then allowed to return to 25° C. The reaction progress was monitored by TLC, and when it was observed that there were no longer any starting materials present, 10 ml of water were added. The reaction solution was then washed with saturated NaCl solution and then dried over sodium sulphate. The solvents were evaporated off under reduced pressure to give 1.7 g (99% yield) of an orange powder.

Characterization

¹H NMR (400 MHz; CDCl₃) δ ppm: 0.87 (t, 3H, CH₃); 1.50 (m, 8H, CH₂); 1.81 (m, 2H, NCH₂CH₂); 3.68 (t, 2H, NHCH₂); 4.17 (m, 4H, NCH₂; CH₂O); 5.26 (s, 1H, NH); 5.77 (d, 1H, C═CH); 6.10 (m, 1H, C═CH); 6.39 (d, 1H, CH═C); 6.70 (d, 1H, ArH); 7.60 (t, 1H, ArH); 8.06 (d, 1H, ArH); 8.43 (d, 1H, ArH); 8.57 (d, 1H, ArH).

### EXAMPLE 4

50 g of ethyl acetate were placed in a 500 ml reactor and then heated to 78° C. for 1 hour. 25 g of methyl methacrylate, 5 g of acrylic acid, 5 g of fluorescent monomer prepared in Example 3, dissolved in 20 g of ethanol, and 0.3 g of 2,5-bis(2-ethylhexanoylperoxy)-2,5-dimethylhexane (Trigonox® 141 from Akzo Nobel) were then added, at 78° C. and over 40 minutes. The mixture was maintained at 78° C. for 1 hour. 15 g of methyl acrylate and 0.2 g of 2,5-bis(2-ethylhexanoylperoxy)-2,5-dimethylhexane were then introduced, at 78° C. and over 40 minutes. The mixture was maintained at 78° C. for 3 hours and was then diluted with 75 g of butyl acetate. The ethyl acetate and the ethanol were distilled off under reduced pressure, 50 g of butyl acetate were then added and 50 g of butyl acetate were distilled off.

A solution comprising 40% polymer solids in butyl acetate was obtained.

This polymer comprised a first block of methyl methacrylate, acrylic acid and monomer disclosed herein, and a second block of methyl acrylate, and also an intermediate segment.

### EXAMPLE 5

An anhydrous foundation comprising the following components (weight %) was prepared:

| | |
|---|---|
| polyethylene wax | 12% |
| volatile silicone oils | 25% |
| phenyl trimethicone | 20% |
| polymethyl methacrylate microspheres | 12% |
| polymer of Example 2 | 6% |
| isododecane | qs 100% |

Preparation:

The waxes were melted and, when it was all clear, the phenyl trimethicone and the silicone oils were added with stirring; the microspheres, the isododecane and the polymer were then added. The mixture was homogenized for 15 minutes and the resulting composition was then cast and allowed to cool.

An anhydrous foundation was obtained.

### EXAMPLE 6

A nail varnish was prepared, comprising:
20% by weight of the polymer obtained in Example 4
qs 100% by weight organic solvents (butyl acetate and ethyl acetate).

### EXAMPLE 7

A stick of lipstick was prepared, comprising the following components (weight %):

| | |
|---|---|
| polyethylene wax | 15% |
| the polymer obtained in Example 2 | 10% AM* |
| hydrogenated polyisobutene (Parléam from Nippon Oil | 25% |
| Fats) | |
| pigments | 10% |
| isododecane | qs 100% |

*AM means active material.

### EXAMPLE 8

100 g of isododecane were placed in a 1 liter reactor and were then heated to 90° C.

18 g of optical-brightening monomer prepared according to Example 1 were dissolved in 90 ml of toluene, followed by addition of 96 g of isobornyl acrylate, 96 g of isobornyl methacrylate, 110 g of isododecane and 1.8 g of initiator, 2,5-bis(2-ethylhexanoylperoxy)-2,5-dimethylhexane (Trigonox® 141 from Akzo Nobel). This mixture was added over 1 hour to the reactor at 90° C. The whole was maintained at 90° C. for 1 hour 30 minutes.

90 g of 2-ethylhexyl acrylate, 90 g of isododecane and 1.2 g of 2,5-bis(2-ethylhexanoylperoxy)-2,5-dimethylhexane were then introduced into the above mixture, at 90° C. and over 30 minutes. The mixture was maintained at 90° C. for 3 hours and was then cooled to 25° C.

After replacing the toluene with isododecane, a solution comprising 46% polymer solids in isododecane was obtained.

The polymer had a weight-average mass (Mw) of 65 100 and a number-average mass (Mn) of 16 000, i.e., a polydispersity index (Ip) of 4.07.

This polymer comprised a first block of isobornyl acrylate, isobornyl methacrylate and optical-brightening monomer disclosed herein, and a second block of 2-ethylhexyl acrylate, and also an intermediate segment.

1. A block polymer comprising at least one first block and at least one second block that are-mutually incompatible, wherein the at least one first block and the at least one second block are linked together via an intermediate segment comprising at least one constituent monomer of the first block and at least one constituent monomer of the second block, wherein at least one of the blocks comprises at least one monomer of formula (I): 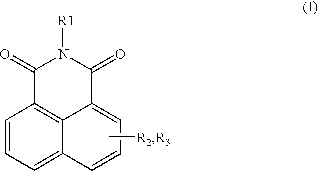
wherein:
R1 is chosen from linear, branched and cyclic, saturated and unsaturated carbon-based radicals comprising from 1 to 32 carbon atoms; optionally substituted with at least one substituent chosen from ═O, OH, NH₂ and halogen atoms; and/or optionally interrupted with at least one hetero atom chosen from O, N, P, Si and S;
R₂ and R₃, which may be identical or different and are present on the same ring or each on a different ring, are each chosen from a hydrogen atom, halogen atoms, and a group of formula —X-G-P₁ (II), with a proviso that at least one of the radicals R₂ and R₃ is a group of formula (II), wherein:
X is chosen from —O—, —S—, —SO—, —SO₂—, —NH— and —NR₄— groups wherein R₄ is chosen from linear, branched and cyclic, saturated and unsaturated carbon-based radicals comprising from 1 to 30 carbon atoms, optionally substituted with at least one substituent chosen from ═O, OH, NH₂ and halogen atoms; and/or optionally interrupted with at least one hetero atom chosen from O, N, P, Si and S;
G is chosen from linear, branched and cyclic, saturated and unsaturated divalent carbon-based radicals comprising from 1 to 32 carbon atoms, optionally substituted with at least one substituent chosen from ═O, OH, NH₂ and halogen atoms; and/or optionally interrupted with at least one hetero atom chosen from O, N, P, Si and S;
P₁ is a polymerizable group chosen from the groups of the following formulae: 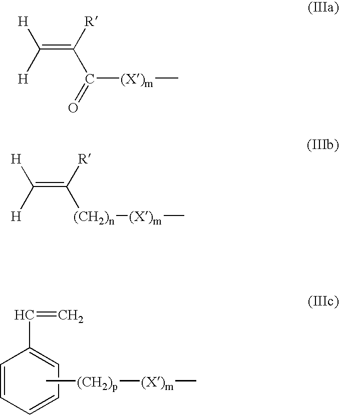
wherein:
R′ is chosen from H and linear and branched, saturated C₁₋₆ hydrocarbon-based radicals,
X′ is chosen from O, NH and NR″ wherein R″ is a radical chosen from C₁₋₆ alkyl, C₆₋₁₀ aryl, (C₆₋₁₀)aryl(C₁₋₆)alkyl and (C₁₋₆)alkyl(C₆₋₁₀)aryl radicals, wherein at least one of the alkyl and aryl groups may be substituted with at least one substituent chosen from OH, halogen atoms, C₁₋₆ alkoxy and C₆₋₁₀ aryloxy groups;
m is equal to 0 or 1; n is equal to 0 or 1; and p is equal to 0, 1 or 2.

2. The block polymer according to claim 1, wherein R1 is chosen from cyclic, linear and branched, saturated and unsaturated hydrocarbon-based radicals, optionally comprising a hydrocarbon-based ring that is itself saturated or unsaturated, comprising from 3 to 18 carbon atoms, and may comprise at least one hetero atom.

3. The block polymer according to claim 2, wherein the at least one hetero atom is chosen from nitrogen, oxygen, and silicon atoms.

4. The block polymer according to claim 1, wherein R1 is chosen from linear, branched and cyclic, saturated and unsaturated hydrocarbon-based radicals comprising from 6 to 13 carbon atoms.

5. The block polymer according to claim 1, wherein R1 is chosen from n-propyl, isopropyl, n-butyl, isobutyl, tert-butyl, n-pentyl, cyclopentyl, n-hexyl, cyclohexyl, n-heptyl, n-octyl, cyclooctyl, decyl, cyclodecyl, dodecyl, cyclododecyl, p-tert-butylcyclohexyl, benzyl and phenyl radicals, and ethyl-2-N-pyrrolidine, (2-methyl)-1-ethylpyrrolidine, 3-propyltriethoxysilane, dialkylpyrimidine, benzothiazyl and fluorenyl radicals.

6. The block polymer according to claim 1, wherein R₂ is a hydrogen atom and R₃ is a group of formula (II).

7. The block polymer according to claim 1, wherein, in the group of formula (II), X is chosen from —O—, —S—, —NH— and —NR₄—.

8. The block polymer according to claim 1, wherein R₄ is chosen from linear, branched and cyclic, saturated and unsaturated hydrocarbon-based radicals optionally comprising a hydrocarbon-based ring that is itself saturated or unsaturated, comprising from 2 to 18 carbon atoms, optionally substituted with at least one substituent chosen from ═O, OH, NH₂ and halogen atoms, and/or is optionally interrupted with at least one hetero atom chosen from O, N, P, Si and S.

9. The block polymer according to claim 8, wherein R₄ is chosen from ethyl, n-propyl, isopropyl, n-butyl, isobutyl, tert-butyl, pentyl, hexyl, cyclohexyl, octyl, decyl, dodecyl, phenyl and benzyl radicals.

10. The block polymer according to claim 1, wherein the divalent radical G is chosen from linear, branched and cyclic, saturated and unsaturated divalent hydrocarbon-based radicals, optionally comprising a hydrocarbon-based ring that is itself saturated or unsaturated, comprising from 2 to 18 carbon atoms, optionally substituted with at least one substituent chosen from ═O, OH, NH₂ and halogen atoms, and/or is optionally interrupted with at least one hetero atom chosen from O, N, P and Si.

11. The block polymer according to claim 1, wherein G is chosen from linear and branched, saturated divalent hydrocarbon-based radicals optionally comprising a saturated hydrocarbon-based ring, comprising in total from 2 to 18 carbon atoms.

12. The block polymer according to claim 1, wherein G is chosen from ethylene, n-propylene, isopropylene (1-methylethylene and 2-methylethylene), n-butylene, isobutylene, pentylene, hexylene, heptylene, cyclohexylene, octylene, decylene, cyclohexyldimethylene, and dodecylene.

13. The block polymer according to claim 1, wherein the polymerizable group P₁ is chosen from the groups of the following formulae: 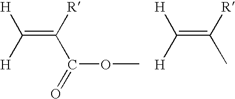
wherein R′ is H or methyl.

14. The block polymer according to claim 1, wherein the monomer of formula (I) is of one of the following formulae:

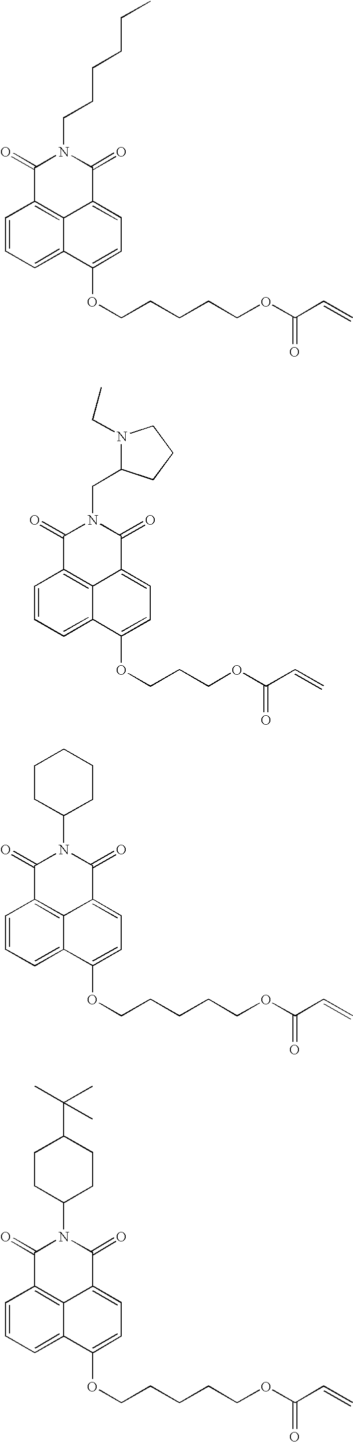

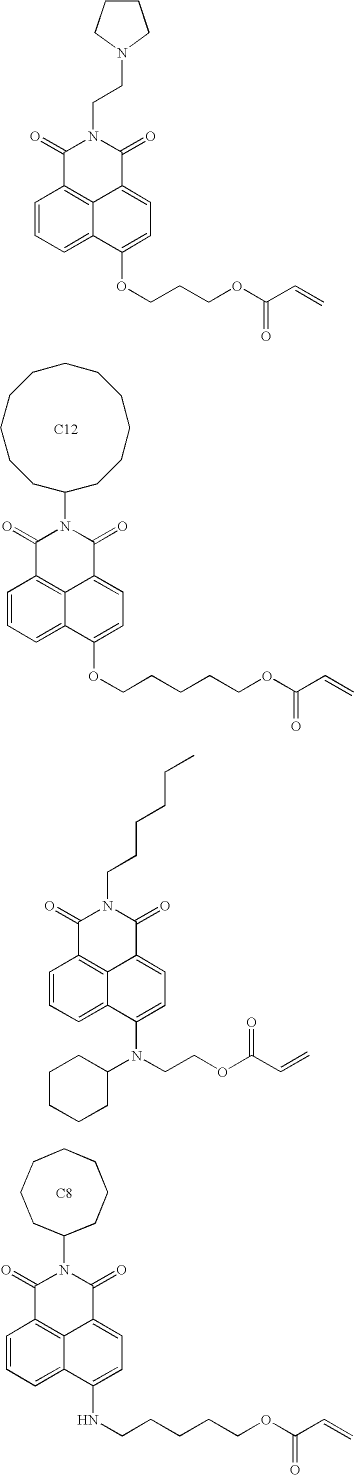

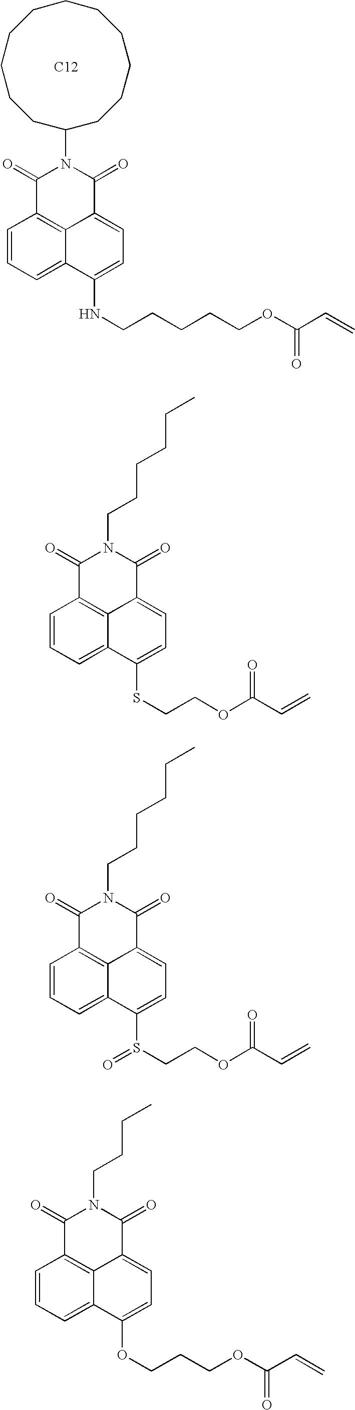

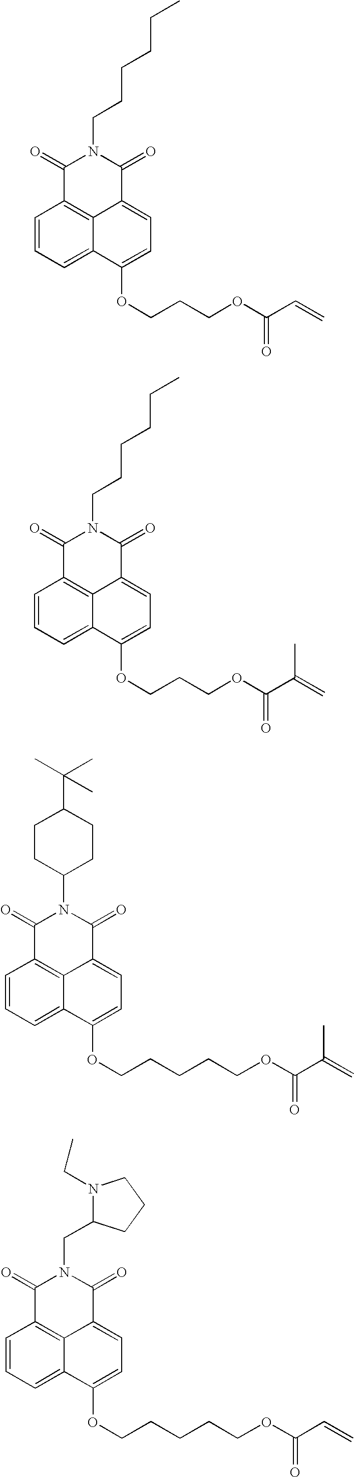

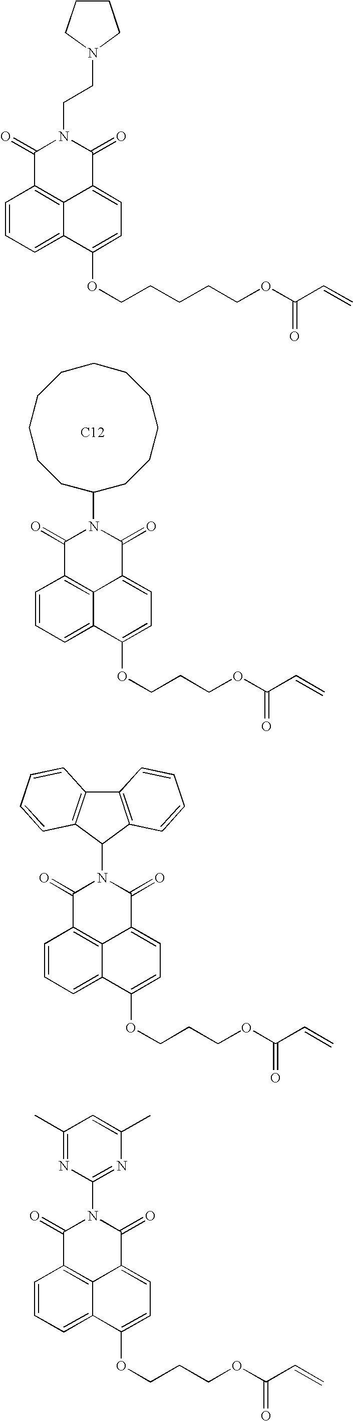

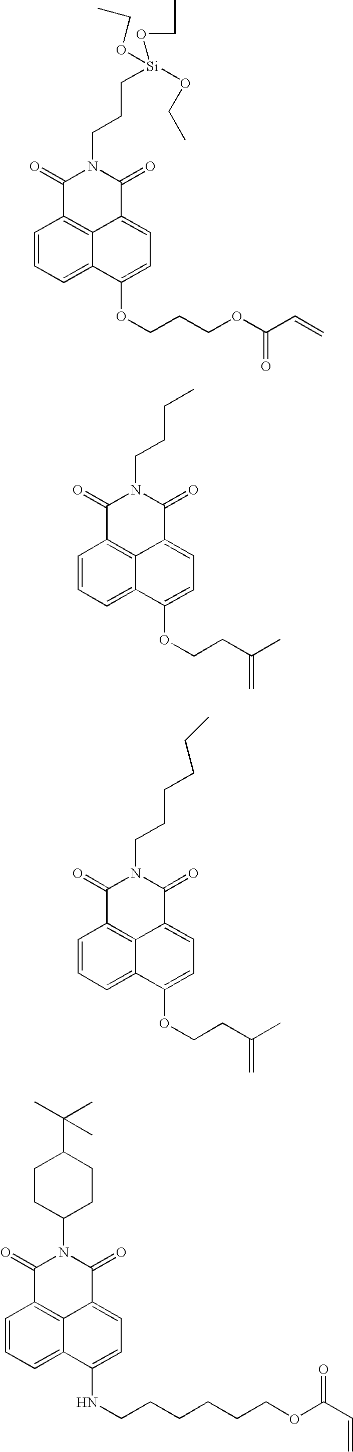

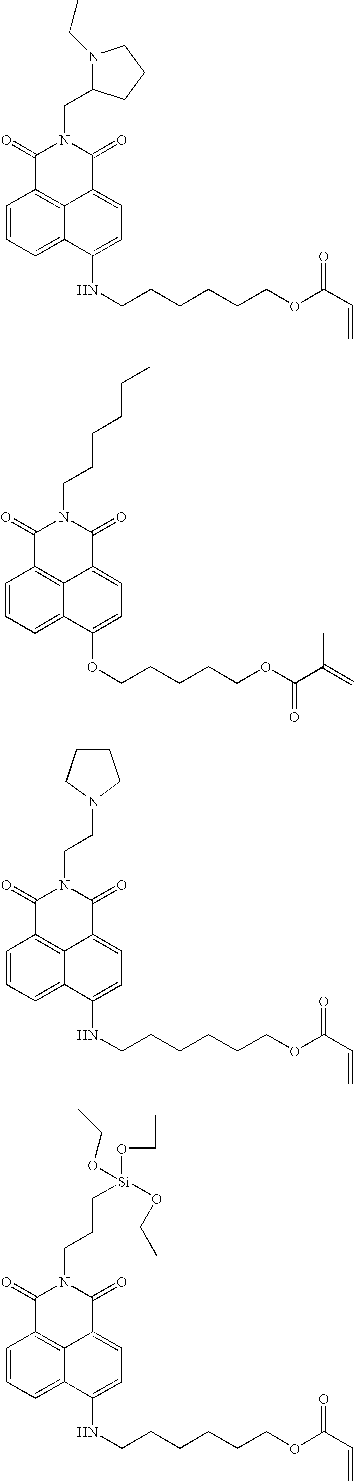

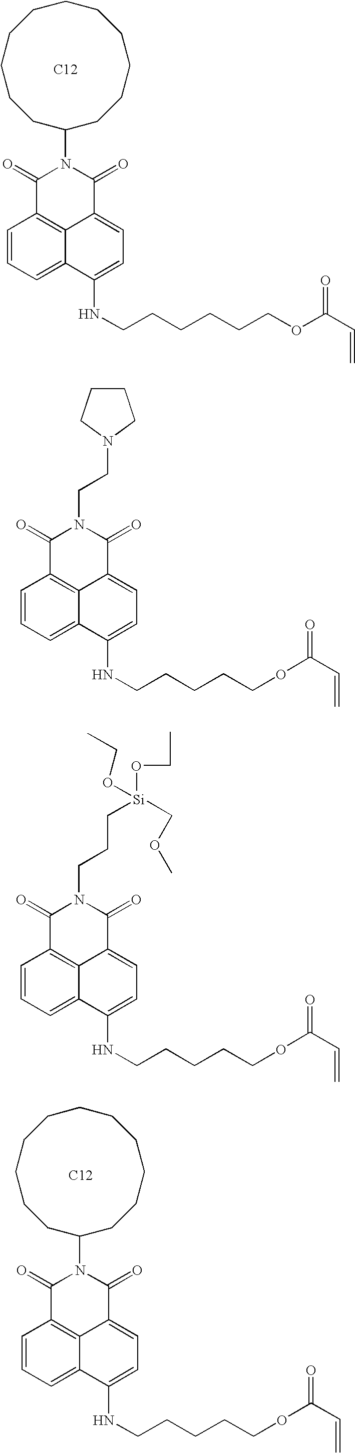

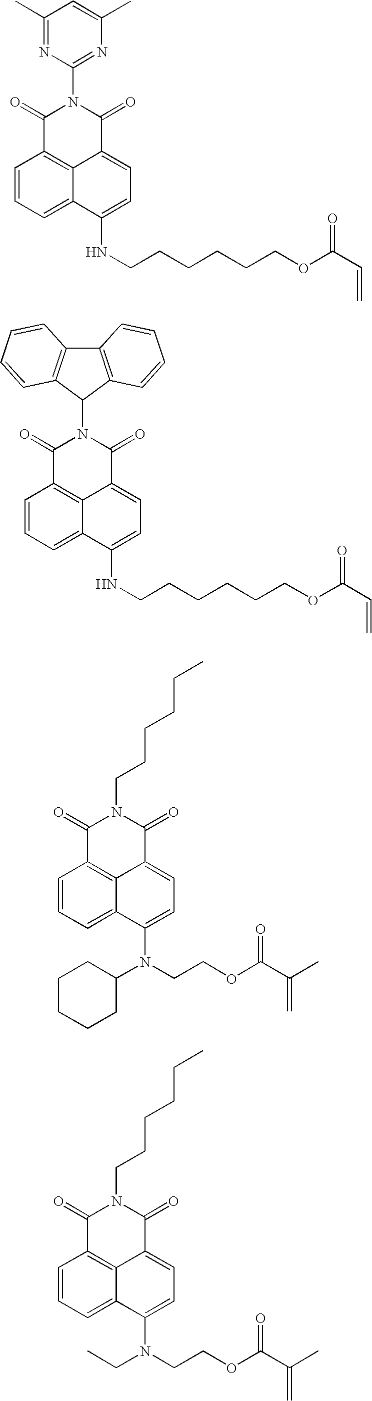

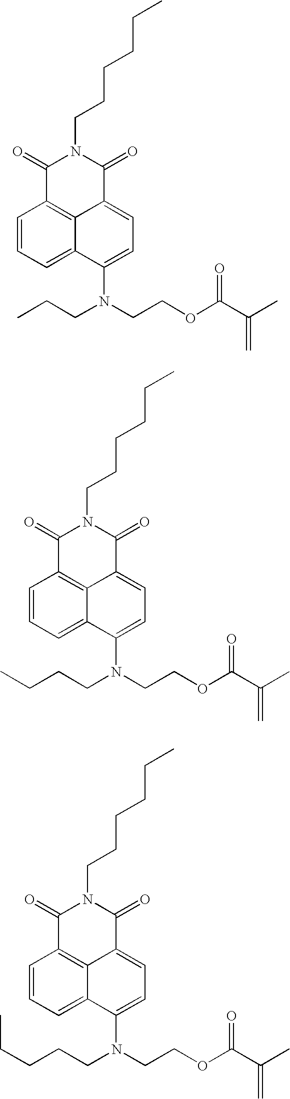

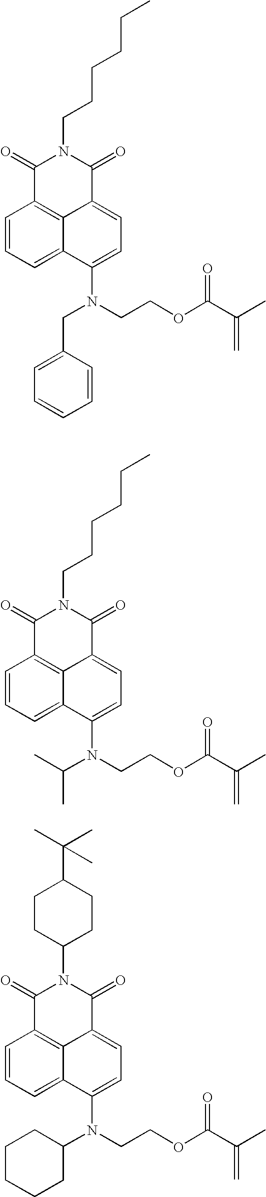

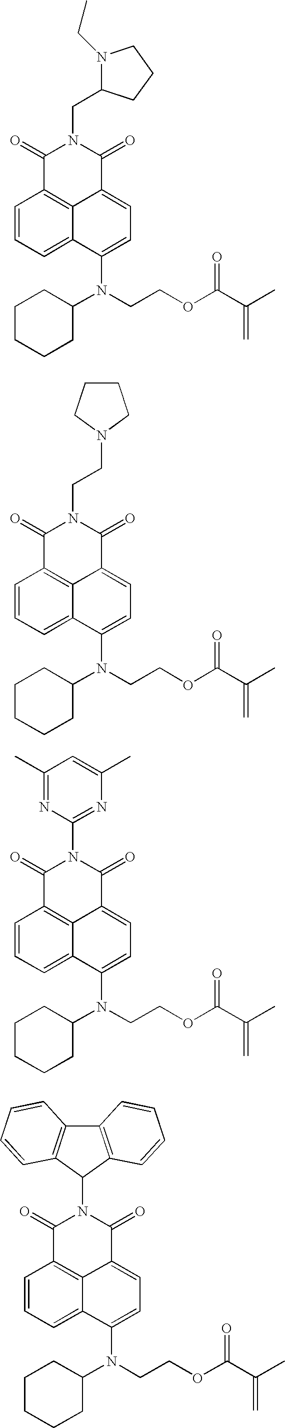

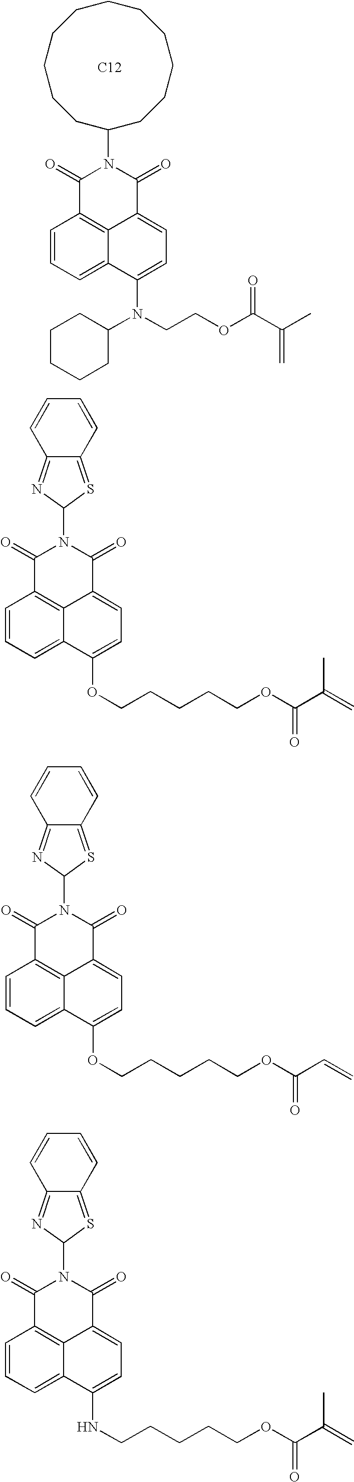

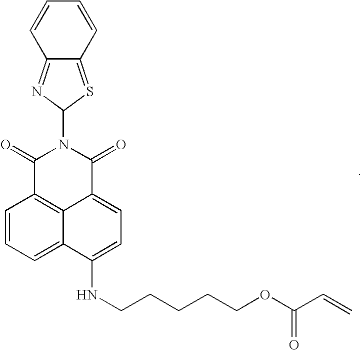

15. The block polymer according to claim 1, further comprising at least one additional comonomer chosen from the following monomers:
(i) ethylenic hydrocarbons comprising from 2 to 10 carbons;
(ii) the (meth)acrylates of formula: 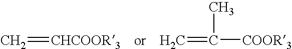
wherein R′₃ is chosen from:
linear and branched alkyl groups comprising from 1 to 18 carbon atoms, in which is optionally intercalated at least one hereto atom chosen from O, N, S and P; wherein the alkyl groups may optionally be substituted with at least one substituent chosen from a hydroxyl group, halogen atoms, and groups Si(R₄R₅), wherein R₄ and R₅, which may be identical or different, are each chosen from C₁ to C₆ alkyl groups and a phenyl group;
C₃ to C₁₂ cycloalkyl groups,
C₃ to C₂₀ aryl groups,
C₄ to C₃₀ aralkyl groups (C₁ to C₈ alkyl groups),
4- to 12-membered heterocyclic groups comprising at least one hetero atom chosen from O, N and S, the ring being aromatic or non-aromatic, and
heterocycloalkyl groups (C₁ to C₄ alkyl groups),
wherein at least one of the cycloalkyl, aryl, aralkyl, heterocyclic and heterocycloalkyl groups may optionally be substituted with at least one substituent chosen from a hydroxyl group, halogen atoms, and linear and branched C₁₋₄ alkyl groups in which is optionally intercalated at least one hetero atom chosen from O, N, S and P, wherein the alkyl groups may optionally be substituted with at least one substituent chosen from a hydroxyl group, halogen atoms, and groups Si(R₄R₅), wherein R₄ and R₅, which may be identical or different, are each chosen from C₁ to C₆ alkyl groups and a phenyl group,
R′₃ may also be a group —(C₂H₄O)ₘ—R″, wherein m=5 to 150 and R″ is chosen from H and C₁ to C₃₀ alkyl groups;
(iii) the (meth)acrylamides of formula: 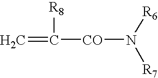
wherein
R₈ is H or methyl; and
R₆ and R₇, which may be identical or different, are each chosen from:
a hydrogen atom; and
linear and branched alkyl groups comprising from 1 to 18 carbon atoms, in which is optionally intercalated at least one hetero atom chosen from O, N, S and P;
wherein the alkyl groups may be optionally substituted with at least one substituent chosen from a hydroxyl group, halogen atoms, and groups Si(R₄R₅), wherein R₄ and R₅, which may be identical or different, are each chosen from C₁ to C₆ alkyl groups and a phenyl group;
C₃ to C₁₂ cycloalkyl groups,
C₃ to C₂₀ aryl groups,
C₄ to C₃₀ aralkyl groups (C₁ to C₈ alkyl groups),
4- to 12-membered heterocyclic groups comprising at least one hetero atom chosen from O, N and S, the ring being aromatic or non-aromatic, and
heterocycloalkyl groups (C₁ to C₄ alkyl groups),
wherein at least one of the cycloalkyl, aryl, aralkyl, heterocyclic and heterocycloalkyl groups may optionally be substituted with at least one substituent chosen from a hydroxyl group, halogen atoms, and linear and branched C₁-C₄ alkyl groups in which is optionally intercalated at least one hetero atom chosen from O, N, S and P, wherein the alkyl groups may optionally be substituted with at least one substituent chosen from a hydroxyl group, halogen atoms, and groups Si(R₄R₅), wherein R₄ and R₅, which may be identical or different, are each chosen from C₁ to C₆ alkyl groups and a phenyl group;
(iv) the vinyl compounds of formula:
CH₂═CH—R₉, CH₂═CH—CH₂—R₉ or CH₂═C(CH₃)—CH₂—R₉
wherein R₉ is chosen from a hydroxyl group, halogen atoms, NH₂, OR₁₀ wherein R₁₀ is chosen from a phenyl group and C₁ to C₁₂ alkyl groups (the monomer is a vinyl or allylic ether); acetamide (NHCOCH₃); a group OCOR₁₁ wherein R₁₁ is chosen from linear and branched alkyl groups comprising from 2 to 12 carbons (the monomer is a vinyl or allylic ester); and a group chosen from:
linear and branched alkyl groups comprising from 1 to 18 carbon atoms, in which is optionally intercalated at least one hetero atom chosen from O, N, S and P; wherein the alkyl groups may optionally be substituted with at least one substituent chosen from a hydroxyl group, halogen atoms, and groups Si(R₄R₅), wherein R₄ and R₅, which may be identical or different, are each chosen from C₁ to C₆ alkyl groups and a phenyl group;
C₃ to C₁₂ cycloalkyl groups,
C₃ to C₂₀ aryl group,
C₄ to C₃₀ aralkyl groups (C₁ to C₈ alkyl groups),
4- to 12-membered heterocyclic groups comprising at least one hetero atom chosen from O, N and S, the ring being aromatic or non-aromatic, and
heterocycloalkyl groups (C₁ to C₄ alkyl groups),
wherein at least one of the cycloalkyl, aryl, aralkyl, heterocyclic and heterocycloalkyl groups may optionally be substituted with at least one substituent chosen from a hydroxyl group, halogen atoms, and linear and branched C₁ to C₄ alkyl groups in which is optionally intercalated at least one hetero atom chosen from O, N, S and P, wherein the alkyl groups may optionally be substituted with at least one substituent chosen from a hydroxyl group, halogen atoms and groups Si(R₄R₅), wherein R₄ and R₅, which may be identical or different, are each chosen from C₁ to C₆ alkyl groups, and a phenyl group;
(v) (meth)acrylic, (meth)acrylamide and vinyl monomers comprising at least one group chosen from fluoro and perfluoro groups;
(vi) silicone-based (meth)acrylic, (meth)acrylamide and vinyl monomers;
(vii) ethylenically unsaturated monomers comprising at least one functional group chosen from carboxylic, phosphoric and sulfonic acid and anhydride functional groups and the salts thereof; and
(viii) ethylenically unsaturated monomers comprising at least one tertiary amine functional group and salts thereof.

16. The block polymer according to claim 15, wherein the at least one additional comonomer is chosen from
(i) ethylene, isoprene, and butadiene;
(ii) the (meth)acrylates of formula: 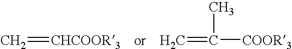
wherein R′₃ is chosen from:
methyl, ethyl, propyl, n-butyl, isobutyl, tert-butyl, hexyl, ethylhexyl, octyl, lauryl, isooctyl, isodecyl, dodecyl, cyclohexyl, t-butylcyclohexyl, and stearyl groups, 2-ethylperfluorohexyl and C₁₋₄ hydroxyalkyl groups, and (C₁₋₄)alkoxy(C₁₋₄)alkyl groups;
an isobornyl group;
a phenyl group;
2-phenylethyl, t-butylbenzyl and benzyl groups;
furfurylmethyl and tetrahydrofurfurylmethyl groups; and
—POE-methyl and —POE-behenyl groups;
(iii) the (meth)acrylamides of formula: 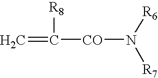
wherein R₆ and R₇, which may be identical or different, are each chosen from
methyl, ethyl, propyl, n-butyl, isobutyl, tert-butyl, hexyl, ethylhexyl, octyl, lauryl, isooctyl, isodecyl, dodecyl, cyclohexyl, t-butylcyclohexyl and stearyl groups, 2-ethylperfluorohexyl and C₁₋₄ hydroxyalkyl groups, and (C₁₋₄)alkoxy(C₁₋₄)alkyl groups;
an isobornyl group;
a phenyl group;
2-phenylethyl, t-butylbenzyl and benzyl groups; and
furfurylmethyl and tetrahydrofurfurylmethyl groups;
(iv) the vinyl compounds of formula:
CH₂═CH—R₉, CH₂═CH—CH₂—R₉ or CH₂═C(CH₃)—CH₂—R₉
wherein R₉ is chosen from
an isobornyl group and cyclohexane;
a phenyl group;
2-phenylethyl, and benzyl groups; and
furfurylmethyl and tetrahydrofurfurylmethyl groups;
(v) ethylperfluorooctyl and 2-ethylperfluorohexyl (meth)acrylates;
(vi) methacryloxypropyltris(trimethylsiloxy)silane and acryloxypropylpolydimethylsiloxane;
(vii) ethylenically unsaturated monomers comprising at least one functional group chosen from functional groups of acrylic acid, methacrylic acid, crotonic acid, maleic anhydride, itaconic acid, fumaric acid, maleic acid, acrylamidopropanesulfonic acid, vinylbenzoic acid and vinylphosphoric acid, and the salts thereof; and
(viii) ethylenically unsaturated monomers comprising at least one tertiary amine functional group chosen from functional groups of 2-vinylpyridine, 4-vinylpyridine, dimethylaminoethyl methacrylate, diethylaminoethyl methacrylate and dimethylaminopropylmethacrylamide and salts thereof.

17. The block polymer according to claim 1, further comprising at least one additional comonomer chosen from the monomers with an optical effect of formulae (A), (B) and (C): 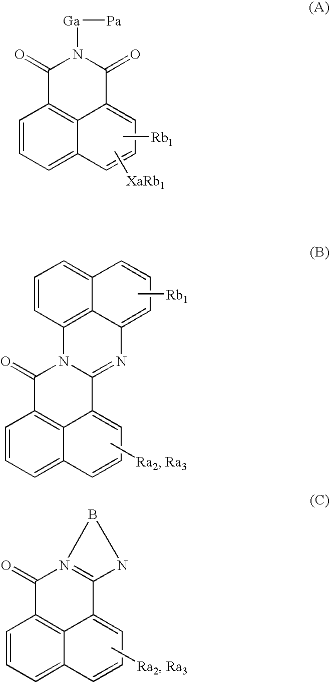
wherein:
Rb₁ is chosen from (i) a hydrogen atom, (ii) halogen atoms, (iii) linear, branched and cyclic, saturated and unsaturated carbon-based radicals comprising from 1 to 12 carbon atoms, optionally substituted with at least one group chosen from ═O, OH and NH₂ and/or optionally interrupted with at least one hetero atom chosen from O, N, P, Si and S; and (iv) a group NRR′ wherein R and R′, which may be identical or different, are each chosen from a hydrogen atom and linear, cyclic and branched, saturated C₁₋₆ hydrocarbon-based radicals;
Ra₂ and Ra₃, which may be identical or different and are present on the same ring or each on a different ring, are each chosen from a hydrogen atom, halogen atoms, and a group of formula -Xa-Ga—Pa (II), with a proviso that at least one of the radicals Ra₂ and Ra₃ is a group of formula (II), wherein:
Xa is chosen from —O—, —S—, —SO—, —SO₂—, —NH— and —NR₄— groups wherein R₄ is chosen from linear, branched and cyclic, saturated and unsaturated carbon-based radicals comprising from 1 to 30 carbon atoms, optionally substituted with at least one substituent chosen from ═O, OH, NH₂ and halogen atoms; and/or optionally interrupted with at least one hetero atom chosen from O, N, P, Si and S;
Ga is chosen from linear, branched and cyclic, saturated and unsaturated divalent carbon-based radicals comprising from 1 to 32 carbon atoms, optionally substituted with at least one substituent chosen from ═O, OH, NH₂ and halogen atoms; and/or optionally interrupted with at least one hetero atom chosen from O, N, P, Si and S;
Pa is a polymerizable group chosen from groups of the following formulae: 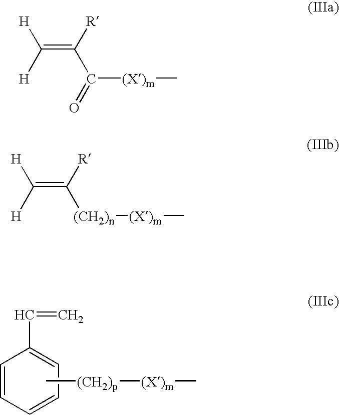
wherein:
R′ is chosen from H and linear and branched, saturated C₁₋₆ hydrocarbon-based radicals,
X′ is chosen from O, NH and NR″ wherein R″ is a radical chosen from C₁₋₆ alkyl, C₆₋₁₀ aryl, (C₆₋₁₀)aryl(C₁₋₆)alkyl and (C₁₋₆)alkyl(C₆₋₁₀)aryl radicals, wherein at least one of the alkyl and aryl groups may be substituted with at least one substituent chosen from OH, halogen atoms, C₁₋₆ alkoxy and C₆₋₁₀ aryloxy groups; and
m is equal to 0 or 1; n is equal to 0 or 1; p is equal to 0, 1 or 2;
B is a group chosen from divalent aromatic groups of formulae (IVa) to (IVd) below: 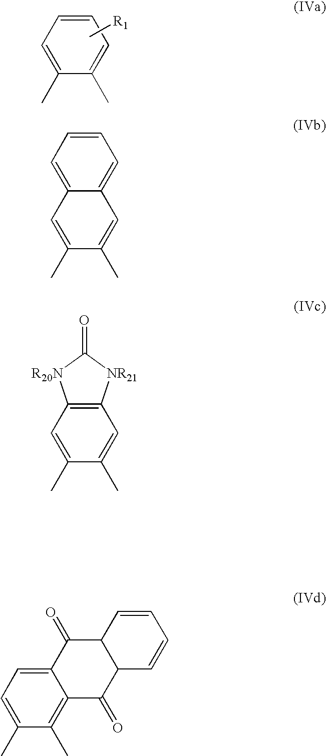
wherein:
R₁ is chosen from linear, branched and cyclic, saturated and unsaturated carbon-based radicals comprising from 1 to 32 carbon atoms, optionally substituted with at least one substituent chosen from ═O, OH, NH₂ and halogen atoms; and
R₂₀ and R₂₁, which may be identical or different, are each chosen from a hydrogen atom, linear and branched C₁₋₈ alkyl radicals, and cyclopentyl, cyclohexyl, cyclooctyl, cyclodecyl, cyclododecyl, benzyl, naphthyl and phenyl radicals.

18. The block polymer according to claim 1, wherein the at least one monomer of formula (I) is present in an amount ranging from 0.01% to 100% by weight, relative to the weight of the block comprising it.

19. The block polymer according to claim 18, wherein the at least one monomer of formula (I) is present in an amount ranging from 1.5% to 30% by weight, relative to the weight of the block comprising it.

20. The block polymer according to claim 1, wherein the at least one monomer of formula (I) is present in an amount ranging from 0.01% to 70% by weight, relative to the total weight of the polymer.

21. The block polymer according to claim 20, wherein the at least one monomer of formula (I) is present in an amount ranging from 1% to 20% by weight, relative to the total weight of the polymer.

22. A composition, comprising, in a physiologically acceptable medium, at least one block polymer comprising at least one first block and at least one second block that are mutually incompatible, wherein the at least one first block and the at least one second block are linked together via an intermediate segment comprising at least one constituent monomer of the first block and at least one constituent monomer of the second block, wherein at least one of the blocks comprises at least one monomer of formula (I): 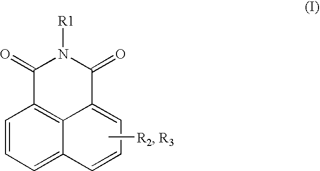
wherein:
R₁ is chosen from linear, branched and cyclic, saturated and unsaturated carbon-based radicals comprising from 1 to 32 carbon atoms; optionally substituted with at least one substituent chosen from ═O, OH, NH₂ and halogen atoms; and/or optionally interrupted with at least one hetero atom chosen from O, N, P, Si and S;
R₂ and R₃, which may be identical or different and are present on the same ring or each on a different ring, are each chosen from a hydrogen atom, halogen atoms, and a group of formula —X-G-P₁ (II), with a proviso that at least one of the radicals R₂ and R₃ is a group of formula (II), wherein:
X is chosen from —O—, —S—, —SO—, —SO₂—, —NH— and —NR₄— groups, wherein R₄ is chosen from linear, branched and cyclic, saturated and unsaturated carbon-based radicals comprising from 1 to 30 carbon atoms, optionally substituted with at least one substituent chosen from ═O, OH, NH₂ and halogen atoms; and/or optionally interrupted with at least one hetero atom chosen from O, N, P, Si and S;
G is chosen from linear, branched and cyclic, saturated and unsaturated divalent carbon-based radicals comprising from 1 to 32 carbon atoms, optionally substituted with at least one substituent chosen from ═O, OH, NH₂ and halogen atoms; and/or optionally interrupted with at least one hetero atom chosen from O, N, P, Si and S;
P₁ is a polymerizable group chosen from the groups of the following formulae: 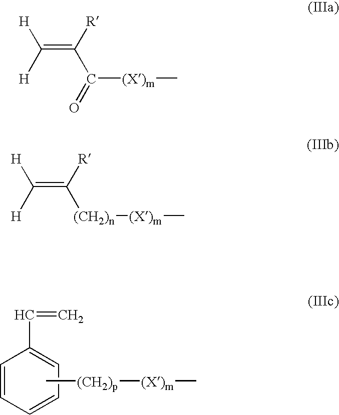
wherein:
R′ is chosen from H and linear and branched, saturated C₁₋₆ hydrocarbon-based radicals,
X′ is chosen from O, NH and NR″ wherein R″ is a radical chosen from C₁₋₆ alkyl, C₆₋₁₀ aryl, (C₆₋₁₀)aryl(C₁₋₆)alkyl and (C₁₋₆)alkyl(C₆₋₁₀)aryl radicals, wherein at least one of the alkyl and aryl groups may be substituted with at least one substituent chosen from OH, halogen atoms, C₁₋₆ alkoxy and C₆₋₁₀ aryloxy groups;
m is equal to 0 or 1; n is equal to 0 or 1; and p is equal to 0, 1 or 2.

23. The composition according to claim 22, wherein the at least one block polymer is present in an amount ranging from 0.01% to 75% by weight, relative to the total weight of the composition.

24. The composition according to claim 23, wherein the at least one block polymer is present in an amount ranging from 6% to 25% by weight, relative to the total weight of the composition.

25. The composition according to claim 22, wherein the physiologically acceptable medium comprises a hydrophilic medium comprising water or a mixture of water and at least one hydrophilic organic solvent and/or comprises a fatty phase.

26. The composition according to claim 25, wherein the fatty phase comprises at least one of waxes, pasty fatty substances, gums, lipophilic organic solvents and oils.

27. The composition according to claim 22, further comprising a particulate phase, which may comprise at least one of pigments, nacres, and fillers.

28. The composition according to claim 22, further comprising at least one dyestuff chosen from water-soluble dyes and liposoluble dyes.

29. The composition according to claim 22, further comprising at least one additional polymer.

30. The composition according to claim 29, wherein the at least one additional polymer is chosen from film-forming polymers.

31. The composition according to claim 22, further comprising at least one ingredient chosen from vitamins, thickeners, gelling agents, trace elements, softeners, sequestering agents, fragrances, acidifying agents, basifying agents, preserving agents, sunscreens, surfactants, antioxidants, agents for preventing hair loss, antidandruff agents, propellants, and ceramides.

32. The composition according to claim 22, wherein the composition is in the form of a suspension; a dispersion; an optionally thickened or even gelled oily solution; an oil-in-water, water-in-oil or multiple emulsion; a gel or a mousse; an oily or emulsified gel; a dispersion of vesicles; a two-phase or multiphase lotion; a spray; a free, compact or cast powder; an anhydrous paste; a lotion, a cream, a salve, a soft paste, an ointment, a cast or moulded solid, and/or a compacted solid.

33. The composition according to claim 22, wherein the composition is in the form of a care and/or makeup product for bodily or facial skin, lips, nails, eyelashes, eyebrows and/or hair, an antisun product, a self-tanning product and/or a hair product for caring for, treating, shaping, making up or coloring the hair.

34. The composition according to claim 22, wherein the composition is in the form of a makeup composition, a makeup rouge or an eyeshadow; a lip product; a concealer product; a blusher, a mascara or an eyeliner; an eyebrow makeup product, a lip pencil or an eye pencil; a nail product; a body makeup product; a hair makeup product chosen from hair mascara and hair lacquer; a protective or care composition for the skin of the face, neck, hands or body; an antisun composition or an artificial tanning composition; and/or a hair product.

35. A cosmetic treatment process for making up or caring for a keratin material, comprising applying to the keratin material a cosmetic composition comprising, in a physiologically acceptable medium, at least one block polymer comprising at least one first block and at least one second block that are mutually incompatible, wherein the at least one first block and the at least one second block are linked together via an intermediate segment comprising at least one constituent monomer of the first block and at least one constituent monomer of the second block, wherein at least one of the blocks comprises at least one monomer of formula (I): 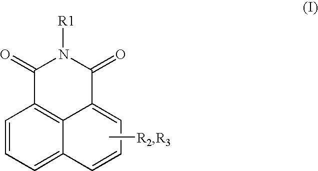
wherein:
R1 is chosen from linear, branched and cyclic, saturated and unsaturated carbon-based radicals comprising from 1 to 32 carbon atoms; optionally substituted with at least one substituent chosen from ═O, OH, NH₂ and halogen atoms; and/or optionally interrupted with at least one hetero atom chosen from O, N, P, Si and S;
R₂ and R₃, which may be identical or different and are present on the same ring or each on a different ring, are each chosen from a hydrogen atom, halogen atoms, and a group of formula —X-G-P₁ (II), with a proviso that at least one of the radicals R₂ and R₃ is a group of formula (II), wherein:
X is chosen from —O—, —S—, —SO—, —SO₂—, —NH— and —NR₄— groups wherein R₄ is chosen from linear, branched and cyclic, saturated and unsaturated carbon-based radicals comprising from 1 to 30 carbon atoms, optionally substituted with at least one substituent chosen from ═O, OH, NH₂ and halogen atoms; and/or optionally interrupted with at least one hetero atom chosen from O, N, P, Si and S;
G is chosen from linear, branched and cyclic, saturated and unsaturated divalent carbon-based radicals comprising from 1 to 32 carbon atoms, optionally substituted with at least one substituent chosen from ═O, OH, NH₂ and halogen atoms; and/or optionally interrupted with at least one hetero atom chosen from O, N, P, Si and S;
P₁ is a polymerizable group chosen from the groups of the following formulae: 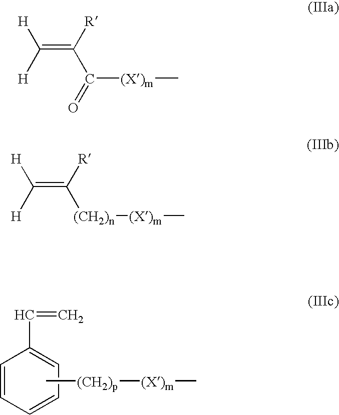
wherein:
R′ is chosen from H and linear and branched, saturated C₁₋₆ hydrocarbon-based radicals,
X′ is chosen from O, NH and NR″ wherein R″ is a radical chosen from C₁₋₆ alkyl, C₆₋₁₀ aryl, (C₆₋₁₀)aryl(C₁₋₆)alkyl and (C₁₋₆)alkyl(C₆₋₁₀)aryl radicals, wherein at least one of the alkyl and aryl groups may be substituted with at least one substituent chosen from OH, halogen atoms, C₁₋₆ alkoxy and C₆₋₁₀ aryloxy groups;
m is equal to 0 or 1; n is equal to 0 or 1; and p is equal to 0, 1 or 2.

36. The cosmetic treatment process according to claim 35, wherein the keratin material is chosen from bodily or facial skin, lips, nails, eyelashes, eyebrows and hair.

37. The cosmetic treatment process according to claim 35, wherein the at least one block polymer is a tensioning agent.

38. The cosmetic treatment process according to claim 35, wherein the cosmetic composition is an anti-wrinkle composition.

39. A nail varnish composition comprising
(1) at least one block polymer comprising at least one first block and at least one second block that are mutually incompatible, wherein the at least one first block and the at least one second block are linked together via an intermediate segment comprising at least one constituent monomer of the first block and at least one constituent monomer of the second block, wherein at least one of the blocks comprises at least one monomer of formula (I): 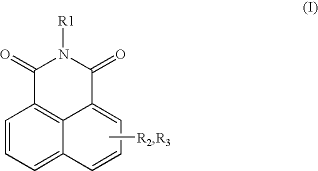
wherein:
R1 is chosen from linear, branched and cyclic, saturated and unsaturated carbon-based radicals comprising from 1 to 32 carbon atoms; optionally substituted with at least one substituent chosen from ═O, OH, NH₂ and halogen atoms; and/or optionally interrupted with at least one hetero atom chosen from O, N, P, Si and S;
R₂ and R₃, which may be identical or different and are present on the same ring or each on a different ring, are each chosen from a hydrogen atom, halogen atoms, and a group of formula —X-G-P₁ (II), with a proviso that at least one of the radicals R₂ and R₃ is a group of formula (II), wherein:
X is chosen from —O—, —S—, —SO—, —SO₂—, —NH— and —NR₄— groups wherein R₄ is chosen from linear, branched and cyclic, saturated and unsaturated carbon-based radicals comprising from 1 to 30 carbon atoms, optionally substituted with at least one substituent chosen from ═O, OH, NH₂ and halogen atoms; and/or optionally interrupted with at least one hetero atom chosen from O, N, P, Si and S;
G is chosen from linear, branched and cyclic, saturated and unsaturated divalent carbon-based radicals comprising from 1 to 32 carbon atoms, optionally substituted with at least one substituent chosen from ═O, OH, NH₂ and halogen atoms; and/or optionally interrupted with at least one hetero atom chosen from O, N, P, Si and S;
P₁ is a polymerizable group chosen from the groups of the following formulae: 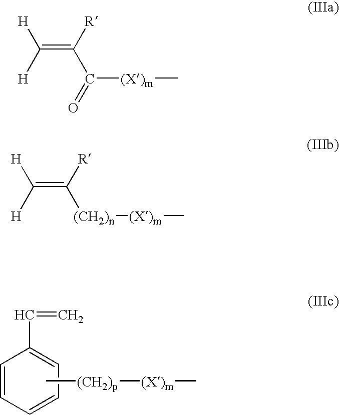
wherein:
R′ is chosen from H and linear and branched, saturated C₁₋₆ hydrocarbon-based radicals,
X′ is chosen from O, NH and NR″ wherein R″ is a radical chosen from C₁₋₆ alkyl, C₆₋₁₀ aryl, (C₆₋₁₀)aryl(C₁₋₆)alkyl and (C₁₋₆)alkyl(C₆₋₁₀)aryl radicals, wherein at least one of the alkyl and aryl groups may be substituted with at least one substituent chosen from OH, halogen atoms, C₁₋₆ alkoxy and C₆₋₁₀ aryloxy groups;
m is equal to 0 or 1; n is equal to 0 or 1; and p is equal to 0, 1 or 2,
(2) at least one organic solvent,
(3) at least one film-forming polymer,
(4) optionally at least one pigment, and
(5) optionally at least one dye.

40. A foundation composition comprising
(1) at least one block polymer comprising at least one first block and at least one second block that are mutually incompatible, wherein the at least one first block and the at least one second block are linked together via an intermediate segment comprising at least one constituent monomer of the first block and at least one constituent monomer of the second block, wherein at least one of the blocks comprises at least one monomer of formula (I): 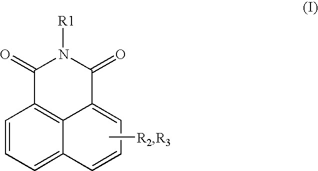
wherein:
R1 is chosen from linear, branched and cyclic, saturated and unsaturated carbon-based radicals comprising from 1 to 32 carbon atoms; optionally substituted with at least one substituent chosen from ═O, OH, NH₂ and halogen atoms; and/or optionally interrupted with at least one hetero atom chosen from O, N, P, Si and S;
R₂ and R₃, which may be identical or different and are present on the same ring or each on a different ring, are each chosen from a hydrogen atom, halogen atoms, and a group of formula —X-G-P₁ (II), with a proviso that at least one of the radicals R₂ and R₃ is a group of formula (II), wherein:
X is chosen from —O—, —S—, —SO—, —SO₂—, —NH— and —NR₄— groups wherein R₄ is chosen from linear, branched and cyclic, saturated and unsaturated carbon-based radicals comprising from 1 to 30 carbon atoms, optionally substituted with at least one substituent chosen from ═O, OH, NH₂ and halogen atoms; and/or optionally interrupted with at least one hetero atom chosen from O, N, P, Si and S;
G is chosen from linear, branched and cyclic, saturated and unsaturated divalent carbon-based radicals comprising from 1 to 32 carbon atoms, optionally substituted with at least one substituent chosen from ═O, OH, NH₂ and halogen atoms; and/or optionally interrupted with at least one hetero atom chosen from O, N, P, Si and S;
P₁ is a polymerizable group chosen from the groups of the following formulae: 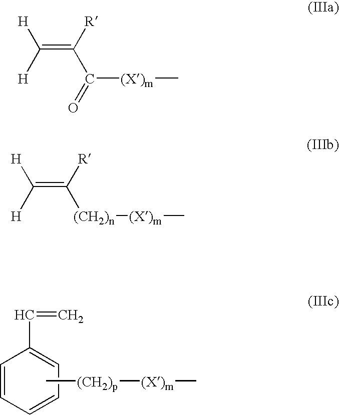
wherein:
R′ is chosen from H and linear and branched, saturated C₁₋₆ hydrocarbon-based radicals,
X′ is chosen from O, NH and NR″ wherein R″ is a radical chosen from C₁₋₆ alkyl, C₆₋₁₀ aryl, (C₆₋₁₀)aryl(C₁₋₆)alkyl and (C₁₋₆)alkyl(C₆₋₁₀)aryl radicals, wherein at least one of the alkyl and aryl groups may be substituted with at least one substituent chosen from OH, halogen atoms, C₁₋₆ alkoxy and C₆₋₁₀ aryloxy groups;
m is equal to 0 or 1; n is equal to 0 or 1; and p is equal to 0, 1 or 2,
(2) at least one oil in a fatty phase,
(3) at least one pigment, and
(4) optionally an aqueous phase.

41. An anti-ageing or anti-wrinkle composition comprising at least one block polymer comprising at least one first block and at least one second block that are mutually incompatible, wherein the at least one first block and the at least one second block are linked together via an intermediate segment comprising at least one constituent monomer of the first block and at least one constituent monomer of the second block, wherein at least one of the blocks comprises at least one monomer of formula (I): 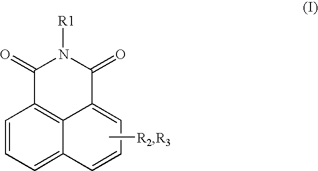
wherein:
R₁ is chosen from linear, branched and cyclic, saturated and unsaturated carbon-based radicals comprising from 1 to 32 carbon atoms; optionally substituted with at least one substituent chosen from ═O, OH, NH₂ and halogen atoms; and/or optionally interrupted with at least one hetero atom chosen from O, N, P, Si and S;
R₂ and R₃, which may be identical or different and are present on the same ring or each on a different ring, are each chosen from a hydrogen atom, halogen atoms, and a group of formula —X-G-P₁ (II), with a proviso that at least one of the radicals R₂ and R₃ is a group of formula (II), wherein:
X is chosen from —O—, —S—, —SO—, —SO₂—, —NH— and —NR₄— groups wherein R₄ is chosen from linear, branched and cyclic, saturated and unsaturated carbon-based radicals comprising from 1 to 30 carbon atoms, optionally substituted with at least one substituent chosen from ═O, OH, NH₂ and halogen atoms; and/or optionally interrupted with at least one hetero atom chosen from O, N, P, Si and S;
G is chosen from linear, branched and cyclic, saturated and unsaturated divalent carbon-based radicals comprising from 1 to 32 carbon atoms, optionally substituted with at least one substituent chosen from ═O, OH, NH₂ and halogen atoms; and/or optionally interrupted with at least one hetero atom chosen from O, N, P, Si and S;
P₁ is a polymerizable group chosen from the groups of the following formulae: 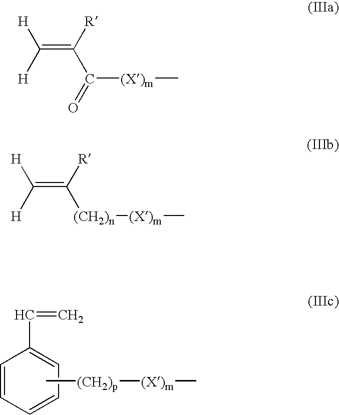
wherein:
R′ is chosen from H and linear and branched, saturated C₁₋₆ hydrocarbon-based radicals,
X′ is chosen from O, NH and NR″ wherein R″ is a radical chosen from C₁₋₆ alkyl, C₆₋₁₀ aryl, (C₆₋₁₀)aryl(C₁₋₆)alkyl and (C₁₋₆)alkyl(C₆₋₁₀)aryl radicals, wherein at least one of the alkyl and aryl groups may be substituted with at least one substituent chosen from OH, halogen atoms, C₁₋₆ alkoxy and C₆₋₁₀ aryloxy groups;
m is equal to 0 or 1; n is equal to 0 or 1; and p is equal to 0, 1 or 2.

42. A cosmetic process for treating wrinkled skin, comprising applying to the wrinkled skin a cosmetic composition comprising, in a cosmetically acceptable medium, at least one block polymer comprising at least one first block and at least one second block that are mutually incompatible, wherein the at least one first block and the at least one second block are linked together via an intermediate segment comprising at least one constituent monomer of the first block and at least one constituent monomer of the second block, wherein at least one of the blocks comprises at least one monomer of formula (I): 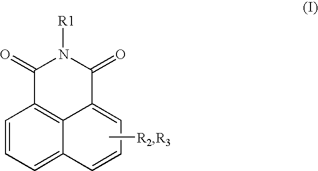
wherein:
R₁ is chosen from linear, branched and cyclic, saturated and unsaturated carbon-based radicals comprising from 1 to 32 carbon atoms; optionally substituted with at least one substituent chosen from ═O, OH, NH₂ and halogen atoms; and/or optionally interrupted with at least one hetero atom chosen from O, N, P, Si and S;
R₂ and R₃, which may be identical or different and are present on the same ring or each on a different ring, are each chosen from a hydrogen atom, halogen atoms, and a group of formula —X-G-P₁ (II), with a proviso that at least one of the radicals R₂ and R₃ is a group of formula (II), wherein:
X is chosen from —O—, —S—, —SO—, —SO₂—, —NH— and —NR₄— groups wherein R₄ is chosen from linear, branched and cyclic, saturated and unsaturated carbon-based radicals comprising from 1 to 30 carbon atoms, optionally substituted with at least one substituent chosen from ═O, OH, NH₂ and halogen atoms; and/or optionally interrupted with at least one hetero atom chosen from O, N, P, Si and S;
G is chosen from linear, branched and cyclic, saturated and unsaturated divalent carbon-based radicals comprising from 1 to 32 carbon atoms, optionally substituted with at least one substituent chosen from ═O, OH, NH₂ and halogen atoms; and/or optionally interrupted with at least one hetero atom chosen from O, N, P, Si and S;
P₁ is a polymerizable group chosen from the groups of the following formulae: 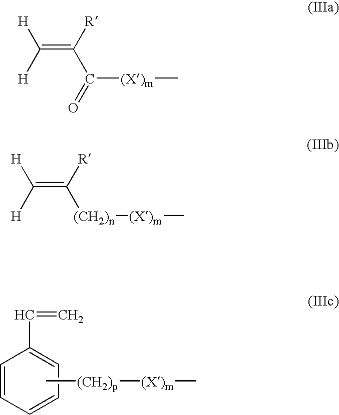
wherein:
R′ is chosen from H and linear and branched, saturated C₁₋₆ hydrocarbon-based radicals,
X′ is chosen from O, NH and NR″ wherein R″ is a radical chosen from C₁₋₆ alkyl, C₆₋₁₀ aryl, (C₆₋₁₀)aryl(C₁₋₆)alkyl and (C₁₋₆)alkyl(C₆₋₁₀)aryl radicals, wherein at least one of the alkyl and aryl groups may be substituted with at least one substituent chosen from OH, halogen atoms, C₁₋₆ alkoxy and C₆₋₁₀ aryloxy groups;
m is equal to 0 or 1; n is equal to 0 or 1; and p is equal to 0, 1 or 2.

43. The cosmetic process according to claim 42, wherein the process is for treating the skin around the eyes.
